(19) United States
(12) Patent Application Publication
(10) Pub. No.: US 2026/0054011 A1
(43) Pub. Date: Feb. 26, 2026

(54) EMERGENCY AUTOMATIC INJECTION DEVICE

(71) Applicant: E3D A.C.A.L., Merom Hagalil (IL)

(72) Inventors: Ziv NAFTALOVITZ, West Galilee (IL); Ilan SHOPEN, Zefat (IL)

(21) Appl. No.: 19/105,484

(22) PCT Filed: Oct. 6, 2023
(86) PCT No.: PCT/IL2023/051066

§ 371 Date: Feb. 21, 2025

Related U.S. Application Data
(60) Provisional application No. 63/414,043, filed on Oct. 7, 2022.

(51) Int. Cl. A61M 5/20; A61M 5/32

(52) U.S. Cl. CPC A61M 5/2033; A61M 5/3202; A61M 2005/3238; A61M 2005/3246

(57) ABSTRACT

An automatic injection device for use with a syringe, including a housing element; at least one resilient element arranged to be located within the housing element; a needle guard slidably positionable with respect to the housing element; a control unit configured to be fixedly coupled with the housing element; a plunger rod biased axially forwardly under an urge of the at least one resilient element and configured to engage the control unit in a storage operative orientation, in which the plunger rod is prevented from forward axial displacement relative to the control unit under the urge of the at least one resilient element; and upon axial rearward displacement of the needle guard with respect to the housing element, the plunger rod is disengaged from the control unit and is permitted to be axially forwardly displaced relative to the control unit under the urge of the at least one resilient element.

### REFERENCE TO RELATED APPLICATIONS

Reference is hereby made to U.S. Provisional Patent Application Ser. No. 63/414,043, filed Oct. 7, 2022 and entitled “EMERGENCY AUTOMATIC INJECTION DEVICE”, the disclosure of which is hereby incorporated by reference in its entirety and priority of which is hereby claimed pursuant to and priority of which is hereby claimed pursuant to 37 CFR 1.78(a) (4) and (5)(i).

### FIELD OF THE INVENTION

The present invention generally relates to an auto injector, and more specifically to an auto-injector adapted for parenteral administration of substances (e.g., a medication) to a living organism (human or animal) by means of pressing the auto injector against an injection site.

### BACKGROUND OF THE INVENTION

Various automatic injectors are known, such that are activatable by means of pressing the automatic injector against an injection site on the skin of a patient. It is important to ensure the needle is protected at all times before, during and after injection of the medicament. It is also required to ensure that the automatic injector is not inadvertently actuated.

### SUMMARY OF THE INVENTION

The present invention seeks to provide an emergency automatic injection device.

There is thus provided in accordance with an embodiment of the present invention or a combination of embodiments thereof, an automatic injection device for use with a syringe including at least one syringe piston and a needle coupled to a forward end thereof, comprising a housing element arranged along a longitudinal axis and having a forward end and a rearward end; at least one resilient element arranged to be located within the housing element; a needle guard slidably positionable with respect to the housing element; a control unit configured to fixedly retain the syringe therewithin and configured to be fixedly coupled with the housing element; a plunger rod biased axially forwardly under an urge of the at least one resilient element and configured to engage the control unit in a storage operative orientation, in which the plunger rod is prevented from forward axial displacement relative to the control unit under the urge of the at least one resilient element; and wherein upon axial rearward displacement of the needle guard with respect to the housing element, the plunger rod is disengaged from the control unit and is permitted to be axially forwardly displaced relative to the control unit under the urge of the at least one resilient element.

Preferably, the automatic injection device also comprising a rear cover, which is fixedly attached to the rearward end of the housing element and wherein a portion thereof is configured to be radially deflected upon the axial rearward displacement of the needle guard. Further preferably, the automatic injection device also comprising a safety cap releasably mounted onto the rear cover and configured to prevent rearward axial displacement of the needle guard up to removal of the safety cap from the rear cover. Still further preferably, the axial rearward displacement of the needle guard is prevented by engagement thereof with the rear cover. Yet further preferably, the axial rearward displacement of the needle guard is allowed upon removal of safety cap from the rear cover. Preferably, the safety cap comprises a rigid rib and at least one resilient spring portion.

In accordance with an embodiment of the present invention, the control unit has at least one locking portion and wherein the plunger rod is configured to engage the at least one locking portion in the storage operative orientation and upon the axial rearward displacement of the needle guard with respect to the housing element, the plunger rod is disengaged from the at least one locking portion.

Preferably, the at least one locking portion is radially deflectable. Further preferably, the automatic injection device also comprising a dampening element between the control unit and the syringe, which is configured to partially absorb the forces created during relative displacement of the plunger rod and the syringe. Still further preferably, the plunger rod has at least one protrusion, which is forwardly supported against the at least one locking portion of the control unit in the storage operative orientation, and wherein the at least one locking portion is outwardly radially supported against a portion of the needle guard and thus prevented from radial outward deflection and from releasing the plunger rod to be forwardly displaced in the storage operative orientation.

Yet further preferably, the at least one resilient spring portion is disposed within openings of the rear cover and wherein inward deflection of the portion of the rear cover is prevented by the rigid rib, which is in turn radially inwardly supported by the plunger rod, thereby preventing rearward axial displacement of the needle guard.

In accordance with an embodiment of the present invention, the at least one resilient spring portion is held within the rear cover and prevent inadvertent removal of the safety cap from the rear cover up to exertion of sufficient axial force, which allows deflection of the at least one resilient spring portion and removal of the safety cap.

Preferably, upon axial rearward displacement of the needle guard relative to the housing element, the portion of the rear cover is being inwardly radially deflected towards the plunger rod. Further preferably, displacement of the needle guard relative to the housing element requires overcoming a predetermined trigger force threshold. Still further preferably, the needle guard has at least one opening; the at least one locking portion is radially supported by the needle guard in the storage operative orientation and upon axial rearward displacement of the needle guard with respect to the housing element, the at least one locking portion is inserted into the at least one opening of the needle guard, thereby actuating the automatic injection device and allowing axial forward displacement of the plunger rod relative to the syringe.

Yet further preferably, the plunger rod has at least one lock engaging portion and wherein the at least one lock engaging portion is forwardly supported by the at least one locking portion in the storage operative orientation and upon axial rearward displacement of the needle guard with respect to the housing element, the at least one locking portion disengages from the at least one lock engaging portion, thereby actuating the automatic injection device and allowing axial forward displacement of the plunger rod relative to the syringe.

In accordance with an embodiment of the present invention, the automatic injection device also comprising a needle shield remover, configured to be releasably coupled to the housing element; and wherein the needle guard engages a portion of the control unit, thereby preventing the needle guard from being axially forwardly displaced relative to the housing element following removal of the needle shield remover from the housing element, and wherein the plunger rod prevents disengagement between the control unit and the needle guard up to axial forward displacement of the plunger rod relative to the syringe.

Preferably, the automatic injection device also comprising a needle shield remover, configured to be releasably coupled to the housing element; and wherein the needle guard engages a portion of the needle shield remover in a storage operative orientation, thereby preventing the needle guard from being axially forwardly displaced relative to the housing element.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element; a control unit configured to fixedly retain the syringe therewithin and configured to be fixedly coupled with the housing element; a plunger rod biased axially forwardly under an urge of the at least one resilient element and configured to engage the control unit in a storage operative orientation, in which the plunger rod is prevented from forward axial displacement relative to the control unit under the urge of the at least one resilient element; and wherein displacement of the needle guard relative to the housing element requires overcoming a predetermined trigger force threshold.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element; a control unit configured to fixedly retain the syringe therewithin and configured to be fixedly coupled with the housing element; a safety cap removably coupled to the automatic injection device and having a rigid portion and a resilient portion; a plunger rod biased axially forwardly under an urge of the at least one resilient element and configured to engage the control unit in a storage operative orientation, in which the plunger rod is prevented from forward axial displacement relative to the control unit under the urge of the at least one resilient element; and wherein axial rearward displacement of the needle guard with respect to the housing element is prevented up until removal of the safety cap from the automatic injection device.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element, the needle guard has at least one opening; a control unit configured to be fixedly coupled with the housing element, the control unit has at least one locking portion; a plunger rod biased axially forwardly under an urge of the at least one resilient element and configured to axially forwardly displace the piston relative to the syringe upon actuation of the automatic injection device; and wherein the locking portion is radially supported by the needle guard in a storage operative orientation and upon axial rearward displacement of the needle guard with respect to the housing element, the at least one locking portion is inserted into the at least one opening of the needle guard, thereby actuating the automatic injection device and allowing axial forward displacement of the plunger rod relative to the syringe.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; a rear cover, which is fixedly attached to the rearward end of the housing element; at least one resilient element arranged to be located within the housing element; a needle guard slidably positionable with respect to the housing element; a control unit configured to fixedly retain the syringe therewithin and configured to be fixedly coupled with the housing element; a plunger rod biased axially forwardly under an urge of the at least one resilient element and configured to axially forwardly displace the piston relative to the syringe upon actuation of the automatic injection device; the plunger rod is further configured to engage the control unit in a storage operative orientation; and wherein axial rearward displacement of the needle guard with respect to the housing element causes a portion of the rear cover to be radially deflected, thereby allowing disengagement between the plunger rod and the control unit, resulting in axial forward displacement of the plunger rod relative to the syringe.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element; a control unit configured to be fixedly coupled with the housing element, the control unit has at least one locking portion; a plunger rod biased axially forwardly under an urge of the at least one resilient element and configured to axially forwardly displace the piston relative to the syringe upon actuation of the automatic injection device; the plunger rod has at least one lock engaging portion and wherein the at least one lock engaging portion is forwardly supported by the at least one locking portion in a storage operative orientation and upon axial rearward displacement of the needle guard with respect to the housing element, the at least one locking portion disengages from the at least one lock engaging portion, thereby actuating the automatic injection device and allowing axial forward displacement of the plunger rod relative to the syringe.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element; a needle shield remover, configured to be releasably coupled to the housing element; a control unit configured to fixedly retain the syringe therewithin and configured to be fixedly coupled with the housing element; a plunger rod biased axially forwardly under an urge of the at least one resilient element; and wherein the needle guard engages a portion of the control unit, thereby preventing the needle guard from being axially forwardly displaced relative to the housing element following removal of the needle shield remover from the housing element, and wherein the plunger rod prevents disengagement between the control unit and the needle guard up to axial forward displacement of the plunger rod relative to the syringe.

In accordance with an embodiment of the present invention, an automatic injection device for use with a syringe including at least one syringe piston and a needle coupled to a forward end thereof, comprising: a housing element arranged along a longitudinal axis and having a forward end and a rearward end; at least one resilient element arranged to be located within the housing element; a needle guard slidably positionable with respect to the housing element; a needle shield remover, configured to be releasably coupled to the housing element; a control unit configured to fixedly retain the syringe therewithin and configured to be fixedly coupled with the housing element; a plunger rod biased axially forwardly under an urge of the at least one resilient element; and wherein the needle guard engages a portion of the needle shield remover in a storage operative orientation, thereby preventing the needle guard from being axially forwardly displaced relative to the housing element.

### BRIEF DESCRIPTION OF THE DRAWINGS

The present invention will be understood and appreciated more fully from the following detailed description, taken in conjunction with the drawings in which:

FIGS. 1A, 1B and 1C are respectively simplified perspective view and two sectional views of an automatic injection device constructed and operative in accordance with an embodiment of the present invention, the sectional views taken along lines B-B and C-C in FIG. 1A;

FIGS. 2A and 2B are respectively a simplified exploded view and a sectional exploded view of the automatic injection device of FIGS. 1A-1C, the sectional view taken along lines B-B in FIG. 2A;

FIGS. 3A, 3B, 3C, 3D and 3E are respectively two simplified perspective views, a simplified side plan view, a simplified top plan view and a simplified sectional view taken along lines E-E in FIG. 3C of a needle shield remover forming part of the automatic injection device of FIGS. 1A-1C;

FIGS. 4A, 4B, 4C and 4D are respectively a simplified perspective view, a simplified side plan view and two sectional views taken along orthogonal lines C-C and D-D in FIG. 4B of a housing element forming part of the automatic injection device of FIGS. 1A-1C;

FIGS. 5A, 5B, 5C, 5D, 5E and 5F are respectively two simplified perspective views, a simplified side plan view, a simplified top plan view and two simplified sectional views taken along lines E-E in FIG. 5C and lines F-F in FIG. 5B of a needle guard forming part of the automatic injection device of FIGS. 1A-1C;

FIGS. 6A, 6B, 6C, 6D and 6E are respectively two simplified perspective views, a simplified side plan view, a simplified top plan view and a simplified sectional view taken along lines E-E in FIG. 6C of a control unit forming part of the automatic injection device of FIG. 1A-1C;

FIGS. 7A, 7B, 7C and 7D are respectively two simplified perspective views, a simplified side plan view and a simplified sectional view taken along lines D-D in FIG. 7C of a syringe assembly forming part of the automatic injection device of FIGS. 1A-1C;

FIGS. 8A, 8B, 8C, 8D and 8E are respectively two simplified perspective views, a simplified side plan view, a simplified top plan view and a simplified sectional view taken along lines E-E in FIG. 8C of a plunger rod forming part of the automatic injection device of FIG. 1A-1C;

FIGS. 9A, 9B, 9C, 9D, 9E and 9F are respectively two simplified perspective views, a simplified side plan view, a simplified top plan view and two simplified sectional views taken along orthogonal lines E-E and F-F in FIG. 9C of a rear cover forming part of the automatic injection device of FIGS. 1A-1C;

FIGS. 10A, 10B, 10C and 10D are respectively two simplified perspective views, a simplified side plan view and a simplified sectional view taken along lines D-D in FIG. 10C of a safety cap forming part of the automatic injection device of FIGS. 1A-1C;

FIGS. 11A, 11B and 11C are simplified drawings of the automatic injection device of FIGS. 1A-10D in a “storage” operative orientation, including a simplified perspective view and two sectional views taken along orthogonal lines B-B and C-C in FIG. 11A;

FIGS. 12A, 12B and 12C are simplified drawings of the automatic injection device of FIGS. 1A-10D in a needle shield removal operative orientation, including a simplified perspective view and two sectional views taken along orthogonal lines B-B and C-C in FIG. 12A, FIG. 12C is partially cut-out to clearly show the internal components of the automatic injection device of FIGS. 1A-10D;

FIGS. 13A and 13B are simplified drawings of the automatic injection device of FIGS. 1A-10D in an intermediate stage of safety cap removal operative orientation, including a simplified perspective view and a sectional view taken along lines B-B in FIG. 13A, FIG. 13B is partially cut-out to clearly show the internal components of the automatic injection device of FIGS. 1A-10D;

FIGS. 14A, 14B and 14C are simplified drawings of the automatic injection device of FIGS. 1A-10D in an end of safety cap removal operative orientation, including a simplified perspective view and two sectional views taken along orthogonal lines B-B and C-C in FIG. 14A;

FIGS. 15A and 15B are simplified drawings of the automatic injection device of FIG. 1A-10D in a needle guard pressing against an injection site operative orientation, including a simplified perspective view and a sectional view taken along lines B-B in FIG. 15A;

FIGS. 16A and 16B are simplified drawings of the automatic injection device of FIG. 1A-10D in a needle insertion operative orientation, including a simplified perspective view and a sectional view taken along lines B-B in FIG. 16A;

FIGS. 17A and 17B are simplified drawings of the automatic injection device of FIG. 1A-10D in an injection initiation operative orientation, including a simplified perspective view and a sectional view taken along lines B-B in FIG. 17A;

FIGS. 18A and 18B are simplified drawings of the automatic injection device of FIG. 1A-10D in an injection operative orientation, including a simplified perspective view and a sectional view taken along lines B-B in FIG. 18A;

FIGS. 19A and 19B are simplified drawings of the automatic injection device of FIG. 1A-10D in an end of injection operative orientation, including a simplified perspective view and a sectional view taken along lines B-B in FIG. 19A;

FIGS. 20A and 20B are simplified drawings of the automatic injection device of FIG. 1A-10D in a removal from injection site operative orientation, including a simplified perspective view and a sectional view taken along lines B-B in FIG. 20A, FIG. 20B is partially cut-out to clearly show the internal components of the automatic injection device of FIGS. 1A-10D;

FIGS. 21A and 21B are simplified drawings of the automatic injection device of FIG. 1A-10D in a needle protection operative orientation, including a simplified perspective view and a sectional view taken along lines B-B in FIG. 21A, FIG. 21B is partially cut-out to clearly show the internal components of the automatic injection device of FIGS. 1A-10D.

### DESCRIPTION OF EMBODIMENTS

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment of the invention in detail, it is to be understood that the invention is not limited in its applications to the details of construction and the arrangement of the components and/or methods set forth in the following description and/or illustrated in the drawings and/or the Examples. The invention can be implemented with other embodiments and can be practiced or carried out in various ways. It is also understood that the phraseology and terminology employed herein is for descriptive purposes and should not be regarded as limiting.

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

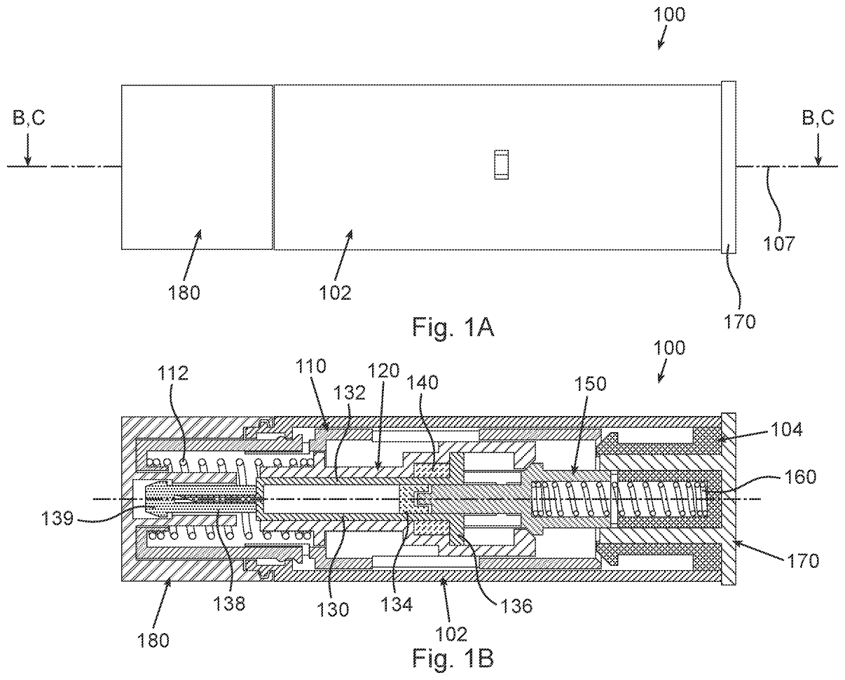

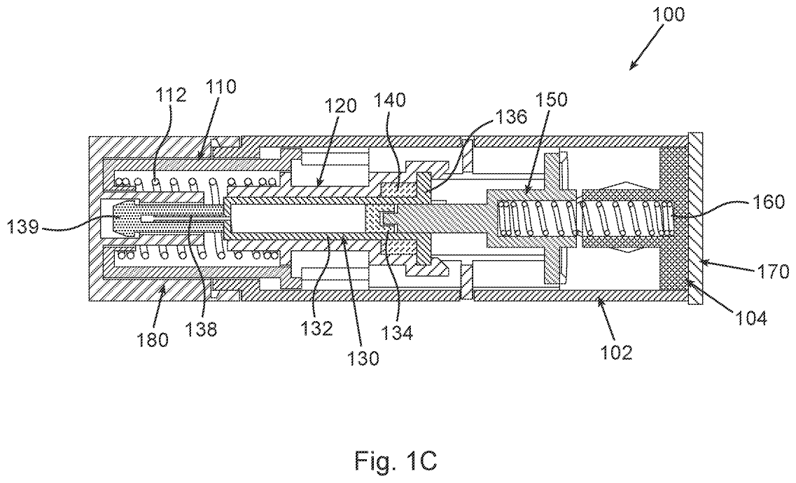

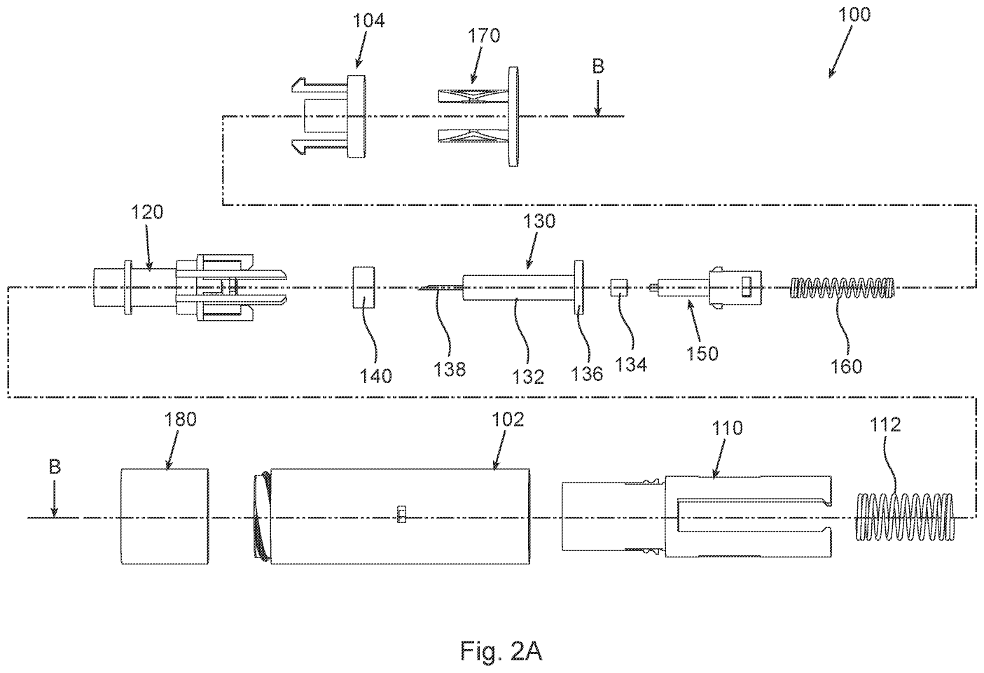

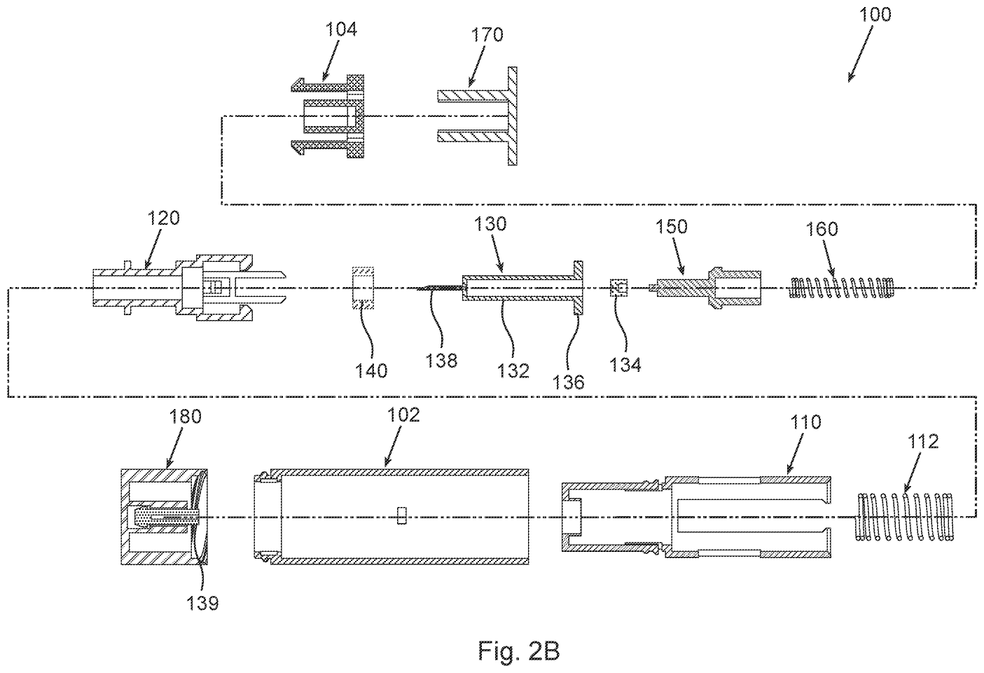

Reference is now made to FIGS. 1A, 1B and 1C, which are respectively simplified perspective view and two sectional views of an automatic injection device constructed and operative in accordance with an embodiment of the present invention, the sectional views taken along lines B-B and C-C in FIG. 1A. Reference is additionally made to FIGS. 2A and 2B, which are respectively a simplified exploded view and a sectional exploded view of the automatic injection device of FIGS. 1A-1C, the sectional view taken along lines B-B in FIG. 2A.

As seen in FIGS. 1A-2B, an automatic injection device 100 comprises a housing element 102 and a rear cover 104, which are preferably fixedly attached, such as by snap-fit engagement. It is noted that the housing element 102 and the rear cover 104 are arranged along a mutual longitudinal axis 107.

Disposed within the enclosure formed by the rear cover 104 and the housing element 102 there is provided a needle guard 110, arranged along the longitudinal axis 107. The needle guard 110 is partially received into the housing element 102 and extends forwardly so as to protrude forwardly from the housing element 102. The needle guard 110 is operative to be biased forwardly under the force of a needle guard spring 112, which is supported between the needle guard 110 and a control unit 120. It is a particular feature of an embodiment of the present invention that the control unit 120 is axially fixed relative to the housing element 102 in all operative orientations of the automatic injection device 100.

A syringe assembly 130 is at least partially received within the control unit 120 and is fixedly attached thereto. The syringe assembly 130 preferably comprises a syringe barrel 132 containing a medicament concealed therewithin by a piston 134. The syringe barrel 132 has a generally circular flange 136 at a rearward end thereof and a needle 138 extending forwardly from a forward end of the syringe barrel 132 and fixedly attached thereto, the needle is preferably covered by a needle shield 139. A dampening element 140 is configured to be supported between the syringe flange 136 and a portion of the control unit 120.

A plunger rod 150 is at least partially inserted into the syringe barrel 132 and is partially received internally to the control unit 120. It is a particular feature of an embodiment of the present invention that the plunger rod 150 is prevented from axial forward displacement along longitudinal axis 107 by engagement with a portion of the control unit 120. The plunger rod 150 is biased forwardly under the urge of an injection spring 160, which is at least partially received into a socket formed within the plunger rod 150. The injection spring 160 is supported between a portion of the plunger rod 150 and the rear cover 104.

It is a particular feature of an embodiment of the present invention that a safety cap 170 is at least partially received by the rear cover 104 and is preferably selectably coupled therewith. When safety cap 170 is coupled to the rear cover 104, rearward axial displacement of the needle guard 110 relative to the housing element 102 is prevented.

It is also seen in FIGS. 1A-2B that a needle shield remover 180 is preferably threadably selectably attached to the housing element 102 and is configured for protecting the needle 138 in storage and for exposing the needle 138 upon detachment of the needle shield remover 180 from the housing element 102, by removing the needle shield 139 covering the needle 138.

The needle guard 110 is configured to be prevented from axial forward displacement relative to the housing element 102 by engagement with the needle shield remover 180 in an initial storage operative orientation of the automatic injection device 100.

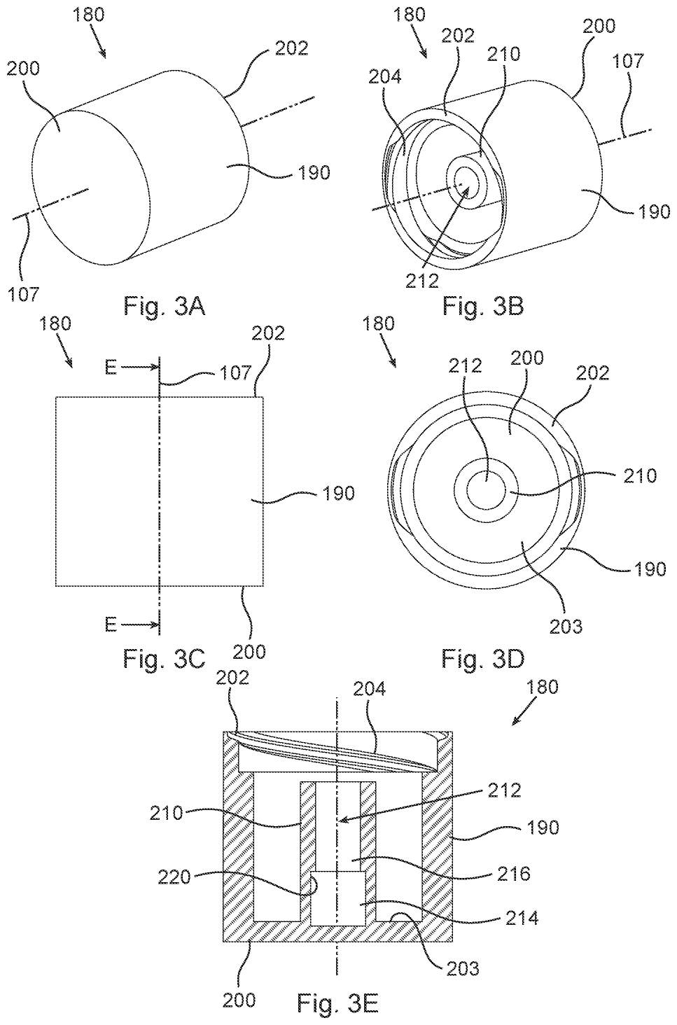

Reference is now made to FIGS. 3A, 3B, 3C, 3D and 3E, which are respectively two simplified perspective views, a simplified side plan view, a simplified top plan view and a simplified sectional view taken along lines E-E in FIG. 3C of the needle shield remover 180 forming part of the automatic injection device 100 of FIGS. 1A-1C.

The needle shield remover 180 preferably is an integrally formed element, preferably injection molded of plastic and is arranged along longitudinal axis of symmetry 107. The needle shield remover 180 has a generally cylindrical portion 190 having a closed forward end 200 and an open rearward end 202. The closed forward end 200 defines a rearwardly facing surface 203. An internally threaded portion 204 is formed on an inner surface of the cylindrical portion 190 and disposed generally adjacent the rearward end 202 thereof.

A needle shield engaging portion 210 extends axially rearwardly from the forward end 200 of the cylindrical portion 190. The needle shield engaging portion 210 is arranged concentrically within the cylindrical portion 190 and arranged along a mutual longitudinal axis therewith. The needle shield engaging portion 210 has a bore 212, which preferably includes a widened bore portion 214 adjacent the forward end 200 of the cylindrical portion 190 and a generally narrower bore portion 216. A forwardly facing shoulder 220 is formed between the bore portions 214 and 216.

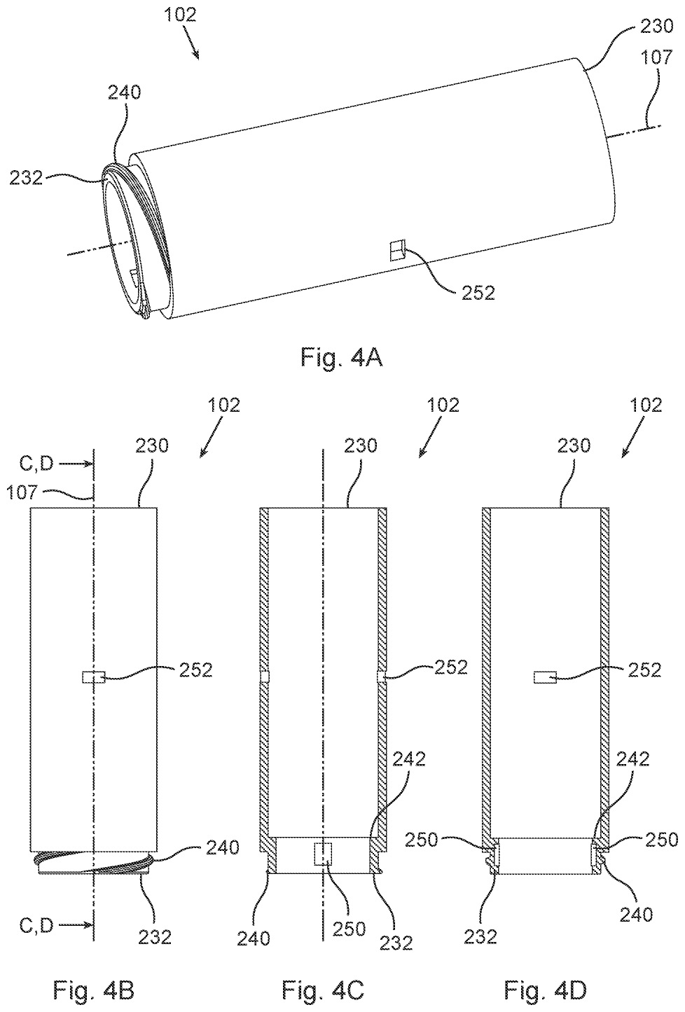

Reference is now made to FIGS. 4A, 4B, 4C and 4D, which are respectively a simplified perspective view, a simplified side plan view and two sectional views taken along orthogonal lines C-C and D-D in FIG. 4B of the housing element 102 forming part of the automatic injection device 100 of FIGS. 1A-1C.

The housing element 102 preferably is an integrally formed element, preferably injection molded of plastic and is arranged along longitudinal axis of symmetry 107.

The housing element 102 is preferably a hollow cylindrical element and has an open rearward end 230 and an open forward end 232. An externally threaded portion 240 is formed on an outer surface of the forward portion of the housing element 102 and disposed generally adjacent the forward end 232 thereof. The externally threaded portion 240 has a slightly smaller diameter than the remaining housing element 102 and thus a rearwardly facing shoulder 242 is formed on the inner surface of the housing element 102, adjacent the forward end 232, which generally extends slightly radially inwardly from the inner surface of the housing element 102.

It is particularly seen in FIGS. 4C & 4D that the threaded portion 240 has at least one opening 250 formed therethrough and disposed generally forwardly of the rearwardly facing shoulder 242. Additionally, typically, a pair of diametrically opposed openings 252 are formed at an intermediate location along the longitudinal extent of the housing element 102.

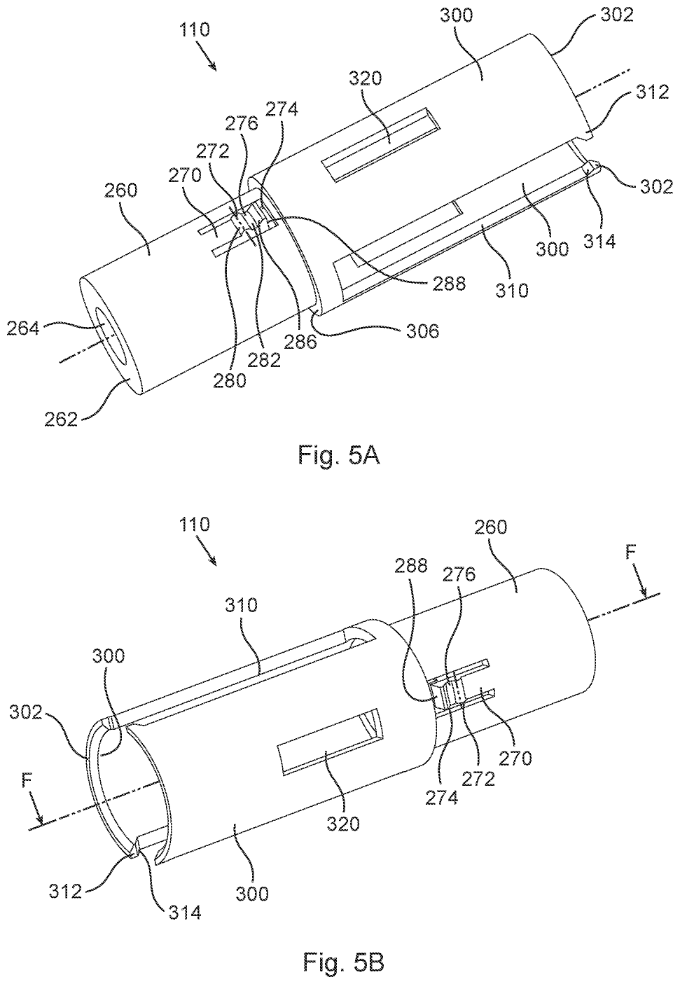

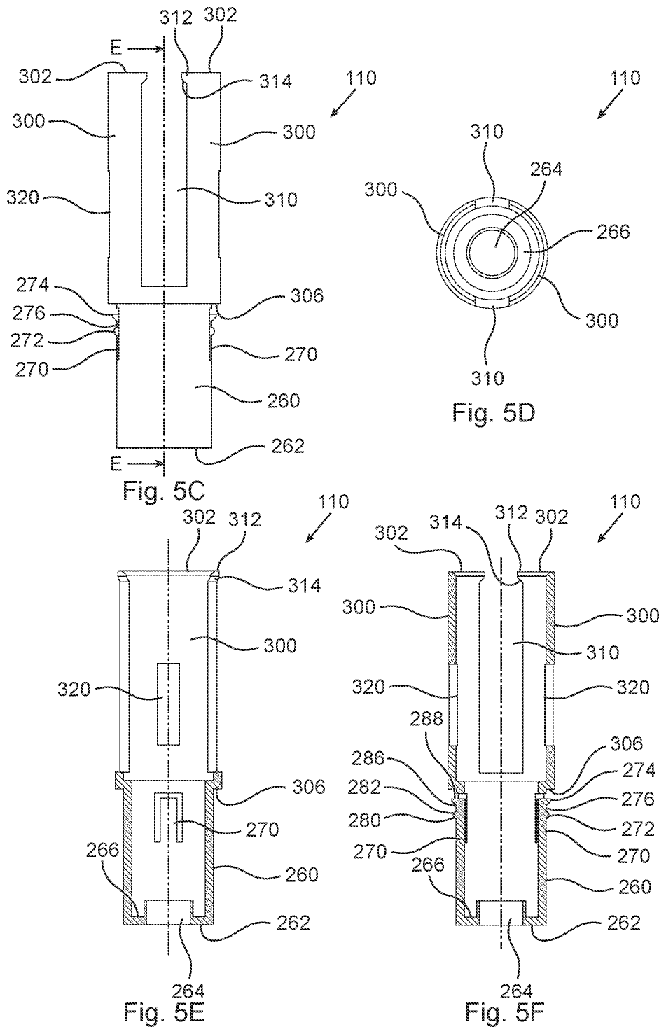

Reference is now made to FIGS. 5A, 5B, 5C, 5D, 5E and 5F, which are respectively two simplified perspective views, a simplified side plan view, a simplified top plan view and two simplified sectional views taken along lines E-E in FIG. 5C and lines F-F in FIG. 5B of the needle guard 110 forming part of the automatic injection device 100 of FIGS. 1A-1C.

The needle guard 110 preferably is an integrally formed element, preferably injection molded of plastic and preferably has a generally cylindrical configuration including a generally tubular forward portion 260, having a forward-facing body engaging surface 262 with an opening 264 formed therethrough. An internal rearwardly facing annular surface 266, located opposite from body engaging surface 262 serves as a spring-seat for spring 112.

Typically, a pair of diametrically opposed snap portions 270 is formed in recesses disposed rearwardly from the forward-facing body engaging surface 262 of the tubular forward portion 260. Each of the snap portions 270 has a pair of outwardly protruding fingers 272 and 274 formed at a rearward end thereof and axially spaced from each other, forming a longitudinal groove 276 therebetween. Finger 272 preferably has a rounded forwardly facing surface 280 and a rounded rearwardly facing surface 282. Finger 274 preferably has a forwardly facing tapered surface 286 and a rearwardly facing shoulder 288, which extends generally transversely with respect to the longitudinal axis 107. The snap portions 270 are configured to be inwardly deflectable. It is noted that the snap portions 270 are configured for operative engagement with the housing element 102.

Needle guard 110 has a pair of side-to-side symmetric mounting arms 300 having rearwardmost ends 302, arranged symmetrically about a longitudinal axis 107. Mounting arms 300 extend rearwardly of tubular portion 260 parallel to longitudinal axis 107. The mounting arms 300 together create an imaginary cylinder having a diameter greater than the diameter of the tubular portion 260, thereby forming a forwardly facing annular shoulder 306 between the tubular portion 260 and the mounting arms 300. A pair of longitudinal grooves 310 are formed between the pair of mounting arms 300, the grooves 310 preferably extend rearwardly from a location generally adjacent the forwardly facing shoulder 306 to the rearwardmost ends 302 of the mounting arms 300. Each one of the rearwardmost ends 302 of mounting arms 300 has a mutually inwardly facing protrusion 312 extending into groove 310 and having a forwardly facing tapered edge 314.

A window 320 is formed in each one of the mounting arms 300. The windows 320 are mutually facing each other and aligned with each other and are adapted for operative engagement with a portion of the control unit 120 in certain operative orientations of the automatic injection device 100.

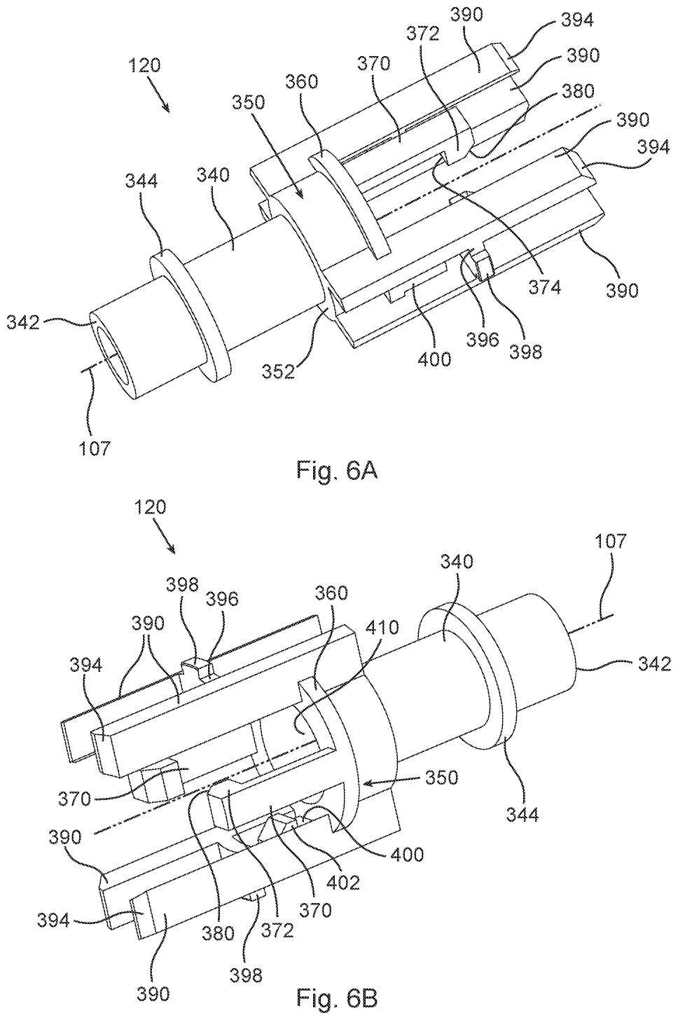

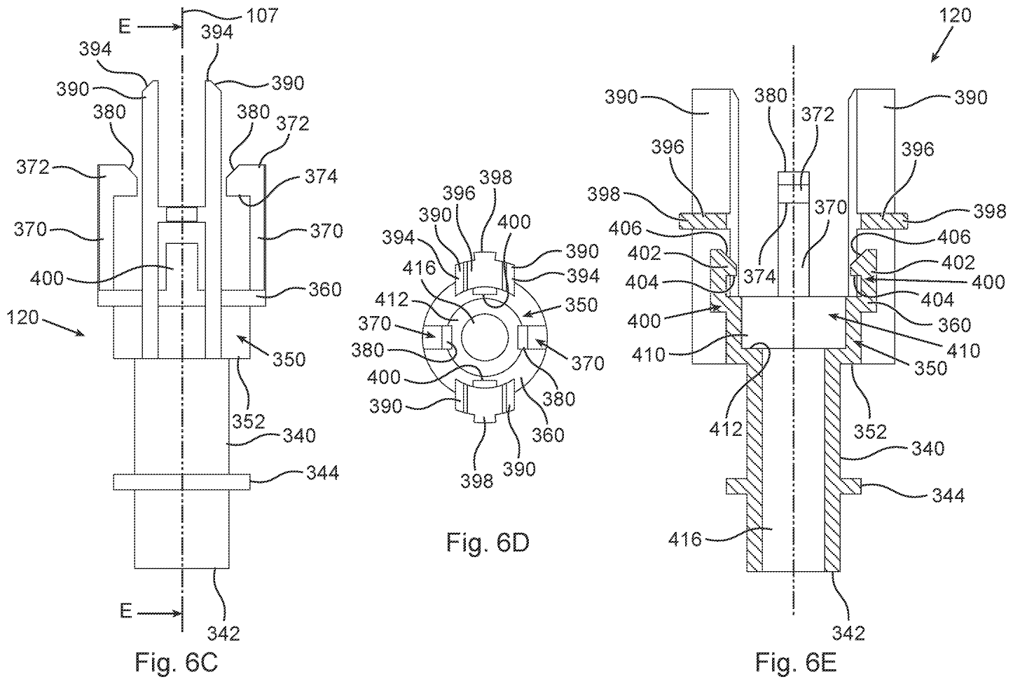

Reference is now made to FIGS. 6A, 6B, 6C, 6D and 6E, which are respectively two simplified perspective views, a simplified side plan view, a simplified top plan view and a simplified sectional view taken along lines E-E in FIG. 6C of the control unit 120 forming part of the automatic injection device 100 of FIGS. 1A-1C.

The control unit 120 preferably is an integrally formed element, preferably injection molded of plastic and is arranged along longitudinal axis of symmetry 107.

The control unit 120 includes a generally hollow cylindric forward portion 340 terminating at a forward annular edge 342 and having an annular flange 344 formed at an intermediate location along the longitudinal extent of the forward portion 340 and extending radially outwardly therefrom.

A bulkhead portion 350 is formed at the rearward end of the forward portion 340, the bulkhead portion 350 is generally circular and has a diameter that is generally greater than the diameter of the forward portion 340, thus forming a forwardly facing shoulder 352 between the forward portion 340 and the bulkhead portion 350. The bulkhead portion 350 extends from the forwardly facing shoulder 352 to a generally widened rearward annular wall 360. Typically, two mutually diametrically opposed locking elements 370 extend rearwardly from the rearward annular wall 360, each terminating at an inwardly radially extending finger 372 defining a forwardly facing surface 374, which extends generally transversely with respect to the longitudinal axis 107, and a rearwardly facing tapered surface 380.

Typically, two pairs of diametrically opposed, radially spaced longitudinal support ribs 390 extend rearwardly from the forwardly facing shoulder 352 to a rearward tapered edge 394. The rearward tapered edges 394 of each one of the pair of support ribs 390 is preferably tapered to an opposite direction. The support ribs 390 within each pair are radially spaced from each other, having a connecting ridge 396 therebetween, the connection ridge 396 has a radially outwardly extending protrusion 398 formed thereon. The support ribs 390 extend radially outwardly from the bulkhead portion 350. Preferably, each pair of supporting ribs 390 is disposed between the two locking elements 370.

It is additionally seen particularly in FIG. 6E that typically two diametrically opposed retaining portions 400 are disposed between the two support ribs 390 in each pair of support ribs 390. The retaining portions 400 extend rearwardly from the annular wall 360 and each preferably has an inwardly projecting finger 402, having a forwardly facing surface 404 and a rearwardly facing tapered surface 406. It is noted that alternatively, the retaining portion 400 can be formed as a continuous ring with an inwardly projecting annular edge.

A socket 410 is formed through the bulkhead portion 350 and the annular wall 360, defining a rearwardly facing shoulder 412. The socket 410 communicates with a through bore 416 extending through the forward portion 340.

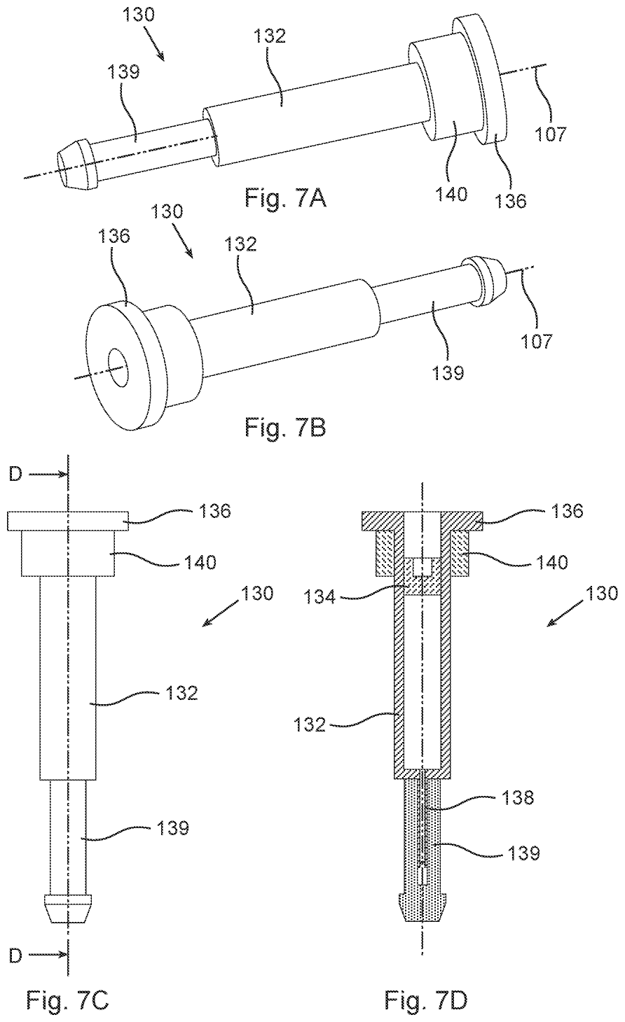

Reference is now made to FIGS. 7A, 7B, 7C and 7D, which are respectively two simplified perspective views, a simplified side plan view and a simplified sectional view taken along lines D-D in FIG. 7C of the syringe assembly 130 forming part of the automatic injection device 100 of FIGS. 1A-1C.

As mentioned hereinabove, with reference to FIGS. 1A-2B, the syringe assembly 130 preferably comprises the syringe barrel 132 containing a medicament concealed therewithin by piston 134. The syringe barrel 132 has a generally circular flange 136 at the rearward end thereof and the needle 138 extends forwardly from the forward end of the syringe barrel 132 and fixedly attached thereto, the needle is preferably covered by needle shield 139. It is seen in FIGS. 7A-7D that the dampening element 140 is mounted onto the syringe barrel 132 adjacent the flange 136 and extending forwardly therefrom. The dampening element 140 is preferably made of a resilient material, such as TPE for example.

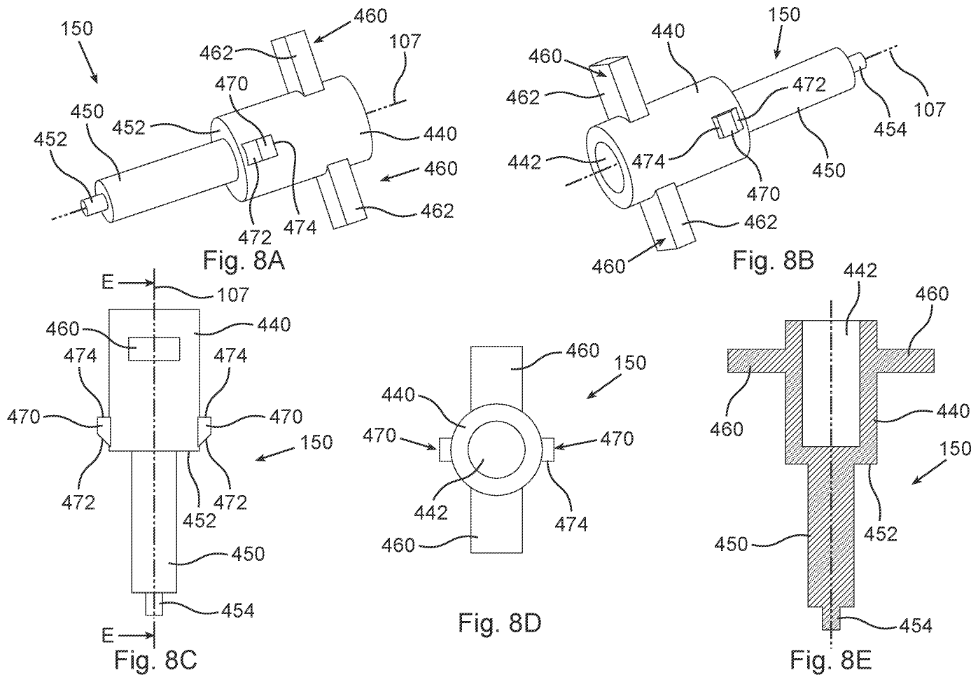

Reference is now made to FIGS. 8A, 8B, 8C, 8D and 8E, which are respectively two simplified perspective views, a simplified side plan view, a simplified top plan view and a simplified sectional view taken along lines E-E in FIG. 8C of the plunger rod 150 forming part of the automatic injection device 100 of FIGS. 1A-1C.

The plunger rod 150 preferably is an integrally formed element, preferably injection molded of plastic and is arranged along longitudinal axis of symmetry 107.

The plunger rod 150 preferably includes a generally hollow cylindrical shaft 440 arranged along longitudinal axis 107 and defining an interior bore 442. A forward shaft portion 450 extends axially forwardly from a forward end 452 of shaft 440 and defining a piston engaging protrusion 454 at a forward end of the forward shaft portion 450.

Typically, two generally diametrically opposed protrusions 460 extend radially outwardly from the shaft 440 and disposed generally adjacent the rearward end thereof. Each one of the protrusions 460 has a side surface 462.

It is also seen in FIGS. 8A-8D that typically two generally diametrically opposed protrusions 470 extend radially outwardly from the shaft 440 and disposed generally adjacent the forward end 452 thereof. The protrusions 470 are generally radially spaced with respect to protrusions 460. Protrusions 470 define a forwardly facing tapered surface 472 and a rearwardly facing surface 474, generally extending transversely with respect to the longitudinal axis 107.

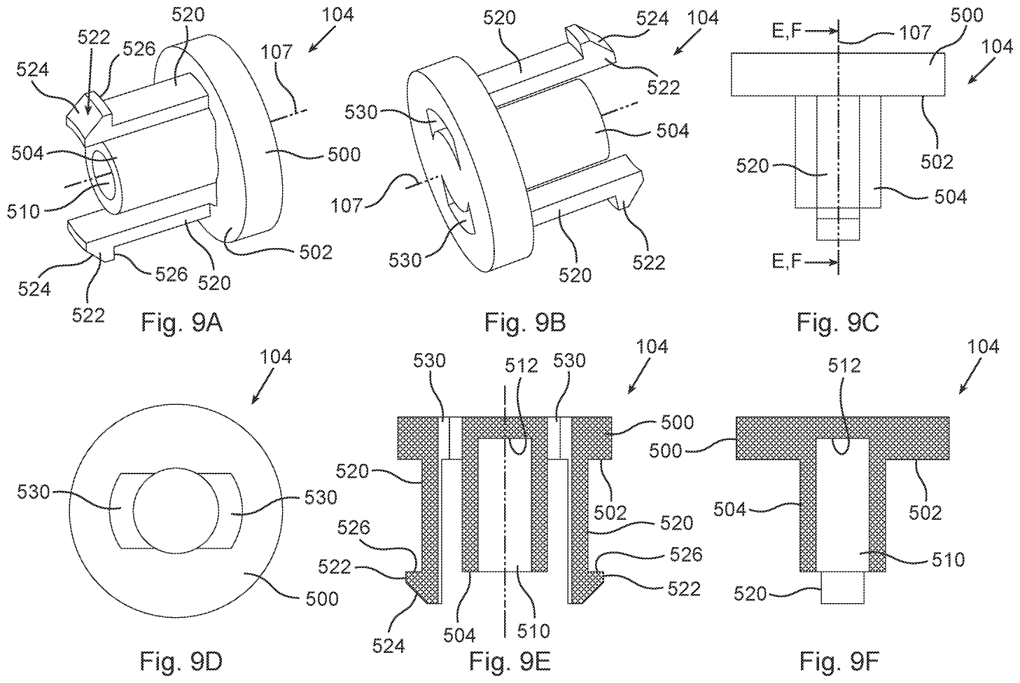

Reference is now made to FIGS. 9A, 9B, 9C, 9D, 9E and 9F, which are respectively two simplified perspective views, a simplified side plan view, a simplified top plan view and simplified two sectional views taken along orthogonal lines E-E and F-F in FIG. 9C of the rear cover 104 forming part of the automatic injection device 100 of FIGS. 1A-1C.

The rear cover 104 preferably is an integrally formed element, preferably injection molded of plastic and arranged along longitudinal axis 107.

The rear cover 104 has a generally circular rearward portion 500 defining a forwardly facing surface 502, from which a hollow cylindrical shaft 504 protrudes axially forwardly. A spring enclosure socket 510 is defined within the shaft 504 and defines a forwardly facing spring seat 512.

Typically, two arms 520 extend forwardly from forwardly facing surface 502 and are externally radially spaced from shaft 504. The arms 520 are preferably diametrically opposed relative to each other. Arms 520 have a radially outwardly extending finger 522 at the forward end thereof. The finger 522 includes a forwardly facing tapered surface 524 and a rearwardly facing surface 526.

Typically, two diametrically opposed openings 530 are formed through the circular rearward portion 500. Preferably, the openings 530 are arcuate in shape.

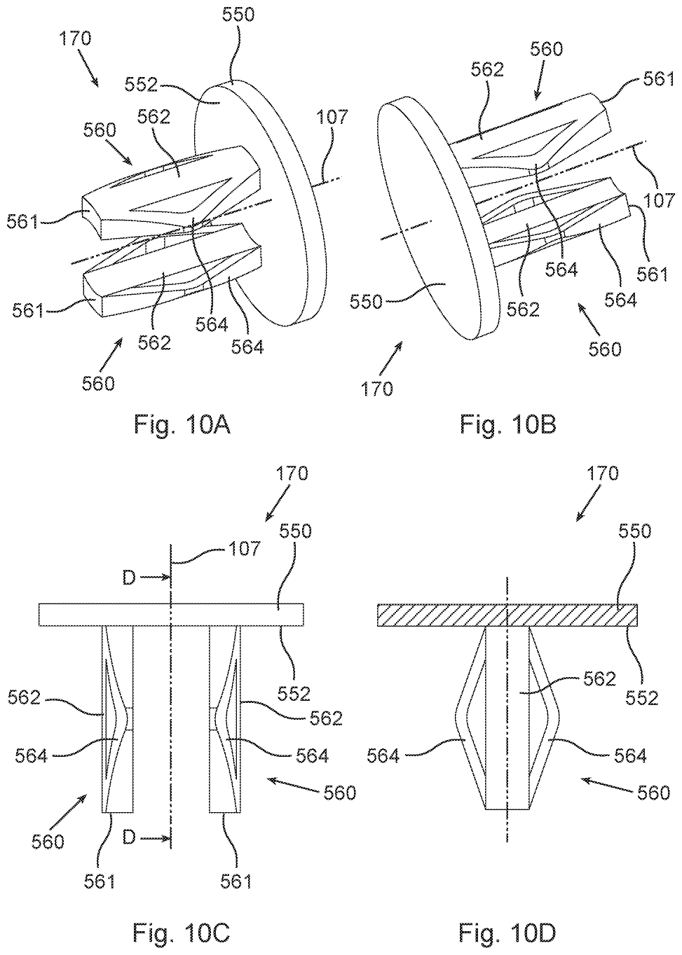

Reference is now made to FIGS. 10A, 10B, 10C and 10D, which are respectively two simplified perspective views, a simplified side plan view and a simplified sectional view taken along lines D-D in FIG. 10C of a safety cap 170 forming part of the automatic injection device 100 of FIGS. 1A-1C.

The safety cap 170 preferably is an integrally formed element, preferably injection molded of plastic and arranged along longitudinal axis 107.

The safety cap 170 has a generally circular rearward portion 550 defining a forwardly facing surface 552. Typically, two restraining arms 560 extend forwardly from forwardly facing surface 552 to a forwardmost edge 561. The restraining arms 560 are preferably diametrically opposed relative to each other and are preferably slightly arcuate in shape. The restraining arms 560 extend about the longitudinal axis 107 and each includes a central rib 562, which is preferably rigid and a pair of leaf spring portions 564, which are preferably resilient and extending tangentially outwardly into two opposite directions from the central rib 562. The leaf spring portions 564 have a triangular shape according to an embodiment of the present invention, alternatively they can have any other shape which provides tangential resiliency to the structure of the restraining arms 560.

The leaf spring portions 564 are seen in their at rest operative orientation in FIGS. 10A-10D, and they are adapted to be tangentially inwardly deflected towards the central rib 562 upon exertion of tangential compressive force thereon.

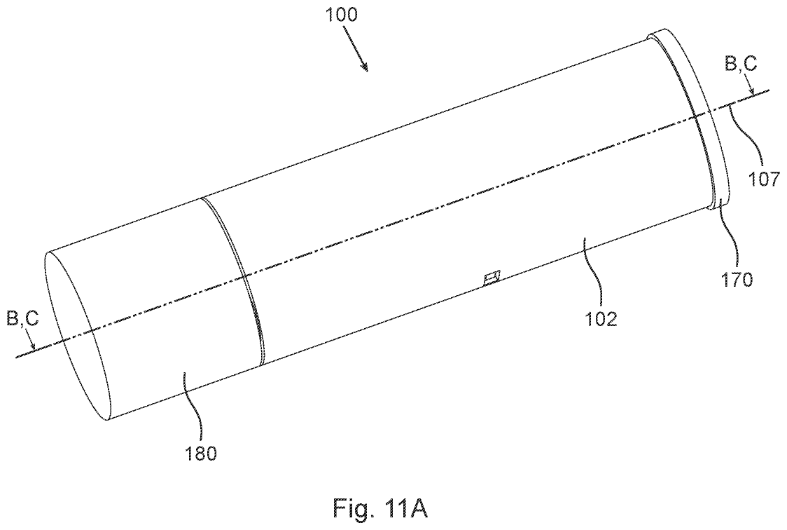

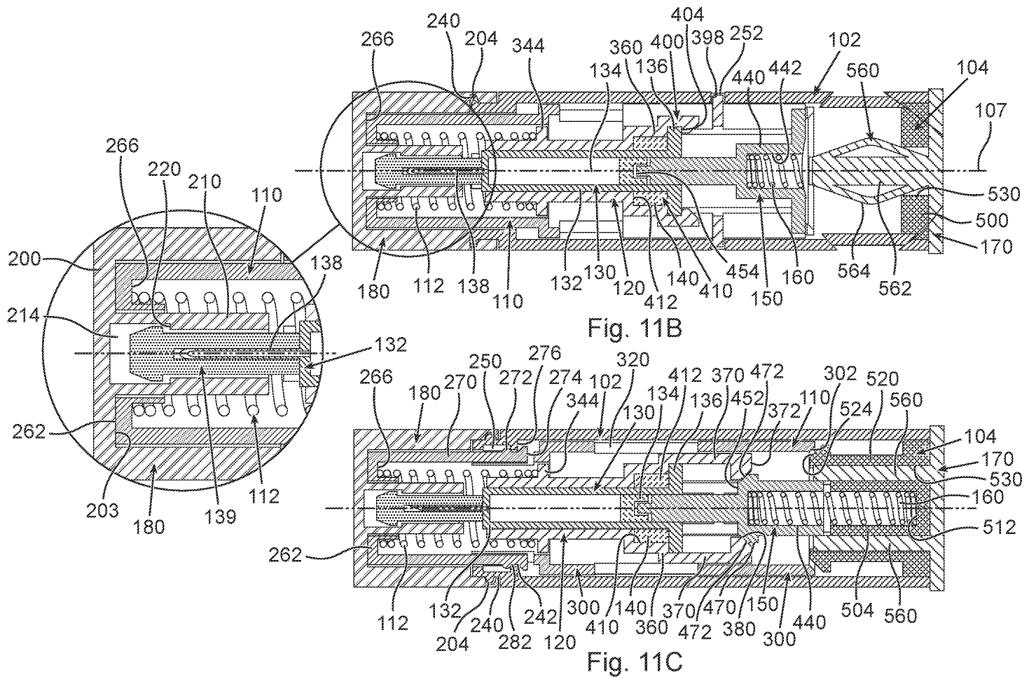

Reference is now made to FIGS. 11A, 11B and 11C, which are simplified drawings of the automatic injection device 100 of FIGS. 1A-10D in a “storage” operative orientation, including a simplified perspective view and two sectional views taken along orthogonal lines B-B and C-C in FIG. 11A.

The automatic injection device 100 comprises the housing element 102, which is fixedly attached to the rear cover 104, both arranged along mutual longitudinal axis 107.

Disposed within the enclosure formed by the rear cover 104 and the housing element 102 is the needle guard 110, which is partially received into the housing element 102 and protrudes forwardly therefrom.

It is seen in FIG. 11C that the needle guard 110 is selectably attached to housing element 102 in this storage operative orientation by means of engagement of outwardly protruding fingers 272 of snap portions 270 of the needle guard 110 with openings 250 of the housing element 102.

Alternatively, the outwardly protruding fingers 272 of the needle guard 110 may be obviated.

The control unit 120 is generally received within the needle guard 110. It is a further particular feature of an embodiment of the present invention that the control unit 120 is fixedly attached to the housing element 102 in all operative orientations of the automatic injection device 100. The outwardly extending protrusions 398 of the control unit 120 are inserted into openings 252 of the housing element 102 and thereby any displacement of the control unit 120 relative to the housing element 102 is prevented.

The syringe assembly 130 is fixedly received within the control unit 120, such that axial displacement between the control unit 120 and the syringe assembly 130 is prevented in all operative orientations of the automatic injection device 100. It is specifically seen that the flange 136 of the syringe barrel 132 is retained by retaining portions 400 of the control unit 120. It is seen specifically in FIG. 11B that the flange 136 is retained between rearward annular wall 360 and forwardly facing surface 404 of retaining portions 400 of the control unit 120.

It is further seen that the dampening element 140 is mounted onto the syringe barrel 132 adjacent the flange 136 and is seated within socket 410 of the control unit 120 to be supported between the syringe flange 136 and the rearwardly facing shoulder 412 of the control unit 120.

It is a particular feature of an embodiment of the present invention that the dampening element 140 slightly protrudes rearwardly from rearward annular wall 360 of the control unit 120, thereby partially absorbing the forces created during relative displacement of the plunger rod 150 and the syringe barrel 132, as described in detail hereinbelow.

The needle guard spring 112 is supported between the control unit 120 and the needle guard 110, specifically between annular flange 344 of the control unit 120 and rearwardly facing annular surface 266 of the needle guard 110.

The needle shield remover 180 is preferably threadably removably coupled to the housing element 102, such that the internally threaded portion 204 of the needle shield remover 180 engages the externally threaded portion 240 of the housing element 102. It is seen that the needle shield 139 covering the needle 138 is partially received within the widened bore portion 214 of the needle shield engaging portion 210 of the needle shield remover 180, such that a portion of the needle shield 139 is disposed in vicinity to the forwardly facing shoulder 220 of the needle shield remover 180. The needle 138 in this storage operative orientation is protected by the needle shield 139 and the needle shield remover 180.

The plunger rod 150 is at least partially inserted into the syringe barrel 132 and is partially received into the control unit 120. In this storage operative orientation, the plunger rod 150 does not yet fully engage the piston 134, thus the piston engaging protrusion 454 of the plunger rod 150 is slightly rearwardly spaced from an internal socket formed within the piston 134.

The plunger rod 150 is biased forwardly under the urge of injection spring 160, which is at least partially received into the interior bore 442 formed within the plunger rod 150. The injection spring 160 is supported between the rearwardly facing surface of the interior bore 442 of the plunger rod 150 and the forwardly facing spring seat 512 of shaft 504 of the rear cover 104.

It is a particular feature of an embodiment of the present invention that the plunger rod 150 is prevented from axial forward displacement along longitudinal axis 107 in this storage operative orientation. The plunger rod 150 is biased axially forwardly under the urge of the injection spring 160, but prevented from such axial forward displacement by engagement with the control unit 120. Specifically, forwardly facing tapered surface 472 of protrusions 470 of the plunger rod 150 are forwardly supported against rearwardly facing tapered surfaces 380 of locking elements 370 of the control unit 120. The locking elements 370 of the control unit 120 are in turn outwardly radially supported against mounting arms 300 of the needle guard 110 and thus are prevented from radial outward deflection and from releasing the plunger rod 150 to be forwardly displaced. The control unit 120 is static relative to the housing element 102, thus the plunger rod 150 is also static relative to the housing element 102 in this storage operative orientation.

The openings 320 within arms 300 of the needle guard 110 are forwardly spaced from the radially extending fingers 372 of the locking elements 370 of the control unit 120 in this storage operative orientation.

It is noted that in this storage operative orientation the forward end 452 of shaft 440 of the plunger rod 150 is rearwardly spaced from the flange 136 of the syringe barrel 132 and the distance between the forward end 452 and the flange 136 defines the amount of medicament to be ejected from the syringe barrel 132.

It is a particular feature of an embodiment of the present invention, as particularly seen in FIGS. 11B & 11C, that rearward axial displacement of the needle guard 110 relative to the housing element 102 is prevented as long as the safety cap 170 is at least partially received within the rear cover 104. Specifically, when leaf spring portions 564 of the restraining arms 560 are disposed forwardly with respect to openings 530 formed in rearward portion 500 of the rear cover 104, the inward deflection of arms 520 of the rear cover 104 is prevented due to the fact that they are radially inwardly supported by the rigid central rib portion 562, which is in turn radially inwardly supported by the shaft 440 of the plunge rod 150 and thus rearward axial displacement of the needle guard 110 is prevented. The leaf spring portions 564 are held within the rear cover 104 and prevent inadvertent removal of the safety cap 170 from the rear cover 104 up to exertion of sufficient axial force, which will allow deflection of the leaf spring portion 564 and removal of the safety cap 170.

It is a further particular feature of an embodiment of the present invention, as particularly seen in FIG. 11C, that the needle guard 110 is prevented from rearward axial displacement relative to the housing element 102 due to engagement of the needle guard 110 and the rear cover 104, which in turn is engaged with the safety cap 170. Particularly, rearwardmost ends 302 of the mounting arms 300 of needle guard 110 are rearwardly supported against forwardly facing tapered surfaces 524 of arms 520 of the rear cover 104. In this storage operative orientation, inward deflection of arms 520 of the rear cover 104 is prevented due to the presence of the safety cap 170 within the automatic injection device 100, thereby preventing rearward axial displacement of the needle guard 110 relative to the housing element 102. It is a particular feature of an embodiment of the present invention, as specifically seen in FIG. 11C, that the safety cap 170 is selectably coupled with the rear cover 104, such that restraining arms 560 of the safety cap 170 are received through openings 530 of the rear cover 104 and protrude forwardly therefrom. The restraining arms 560 of the safety cap 170 are disposed radially outwardly of the shaft 504 of the rear cover and shaft 440 of the plunger rod 150 and radially inwardly of arms 520 of the rear cover 104, thereby preventing inward deflection of the arms 520 under axial rearward displacement by the needle guard 110. The leaf spring portions 564 of the restraining arms 560 of the safety cap 107 keep the safety cap 170 coupled with the rear cover 104, since the leaf spring portions 564 are wider than the openings 530 of the rear cover 104. The rib 562 of the restraining arms 560 applies radial force on the shaft 440 of the plunger rod 150 to prevent inward deflection of the arms 520 of the rear cover 104.

The needle guard 110 is biased to be axially displaced forwardly under the force of the needle guard spring 112. It is seen in FIGS. 11B & 11C that the axial forward displacement of the needle guard 110 is prevented due to engagement of the needle guard 110 and the needle shield remover 180 in this storage operative orientation, specifically by engagement of the forward facing body engaging surface 262 of the needle guard 110 with the rearwardly facing surface 203 of the needle shield remover 180.

It is a particular feature of an embodiment of the present invention that the rearward portion 550 of the safety cap 170 has a closed end with no openings and the forward end 200 of needle shield remover 180 has a closed end with no openings. The lack of openings at the forward end of the automatic injection device 100 is beneficial from usability perspective, as user confusion in terms of premature injection before removal of the needle shield remover 180 can be prevented.

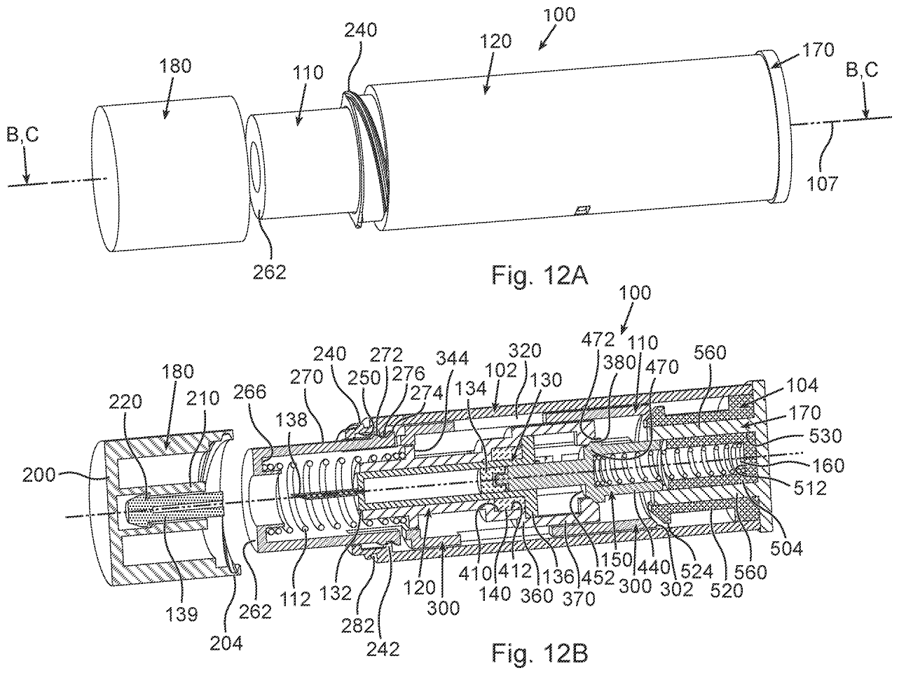

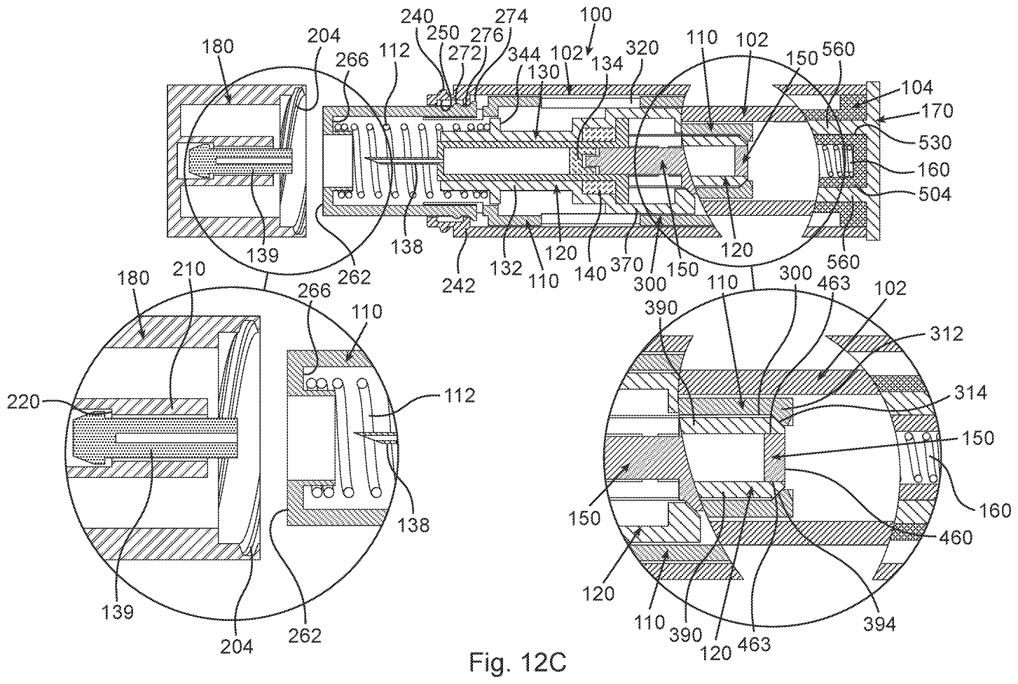

Reference is now made to FIGS. 12A, 12B and 12C, which are simplified drawings of the automatic injection device 100 of FIGS. 1A-10E in a needle shield removal operative orientation, including a simplified perspective view and two sectional views taken along orthogonal lines B-B and C-C in FIG. 12A, FIG. 12C is partially cut-out to clearly show the internal components of the automatic injection device 100 of FIGS. 1A-10D.

It is appreciated that all spatial relationships between the various components of the automatic injection device 100 remain the same as described hereinabove with respect to the storage operative orientation illustrated in FIGS. 11A-11C, besides the following spatial relationships:

The user grips the needle shield remover 180 and preferably threadably removes it from the housing element 102, by means of engagement between the internally threaded portion 204 of the needle shield remover 180 and the externally threaded portion 240 of the housing element 102, and thereby removes the needle shield 139 to expose needle 138 of the syringe assembly 130.

Following removal of the needle shield removal 180, the forwardly facing body engaging surface 262 of the needle guard 110 is exposed and protrudes forwardly from the housing element 102 to a first longitudinal extent.

It is a particular feature of an embodiment of the present invention that once the needle shield remover 180 is detached from the housing element 102, the needle guard 110 is biased axially forwardly by needle guard spring 112 and is prevented from axial forward displacement by means of engagement with a portion of the control unit 120, which is in turn prevented from deflection by engagement with a portion of the plunger rod 150. It is specifically seen in FIGS. 12B & 12C that the needle guard 110 is selectably attached to the housing element 102 in this needle shield removal operative orientation by means of engagement of the outwardly protruding fingers 272 of snap portions 270 of the needle guard 110 with openings 250 of the housing element 102. Alternatively, the outwardly protruding fingers 272 of the needle guard 110 may be obviated.

It is specifically seen in FIGS. 12B & 12C that the groove 276 of the needle guard 110 is generally disposed in front of the rearwardly facing shoulder 242 of the housing element 102, such that the outwardly protruding finger 272 is disposed forwardly of the rearwardly facing shoulder 242 and the outwardly protruding finger 274 is disposed rearwardly of the rearwardly facing shoulder 242.

It is further specifically seen in FIG. 12C that the inwardly facing protrusions 312 of the needle guard 110 are generally axially forwardly supported by support ribs 390 of the control unit 120, such that forward axial displacement of the needle guard 110 relative to the housing element 102 is prevented. As specifically seen in FIG. 12C, the forwardly facing tapered edges 314 of inwardly facing protrusions 312 of the needle guard 110 are forwardly supported against rearward tapered edges 394 of two support ribs 390 of the control unit 120 and the two support ribs 390 of the control unit 120 are prevented from inward deflection towards each other under the axial forward bias of the needle guard 110 due to the fact that protrusions 460 of the plunger rod 150 are seated between each pair of supporting ribs 390 of the control unit 120, such that side surfaces 463 of protrusions 460 of the plunger rod 150 engage the support ribs 390 and serve as a wedge therebetween to prevent their inward deflection.

It is also particularly seen in FIG. 12C that the support ribs 390 of the control unit 120 are outwardly radially supported by the mounting arms 300 of the needle guard 110, which are in turn radially outwardly supported by the housing element 102.

It is a particular feature of an embodiment of the present invention that rearward axial displacement of the needle guard 110 relative to the housing element 102 is prevented as long as the safety cap 170 is at least partially received within the rear cover 104. Specifically, when leaf spring portions 564 of the restraining arms 560 are disposed forwardly with respect to openings 530 formed in rearward portion 500 of the rear cover 104, the inward deflection of arms 520 of the rear cover 104 is prevented and thus rearward axial displacement of the needle guard 110 is prevented.

It is a further particular feature of an embodiment of the present invention, as particularly seen in FIG. 12B, that the needle guard 110 is still prevented from rearward axial displacement relative to the housing element 102 in this needle shield removal operative orientation due to engagement of the needle guard 110 and the rear cover 104, which in turn is engaged with the safety cap 170. Particularly, rearwardmost ends 302 of the mounting arms 300 of needle guard 110 are rearwardly supported against forwardly facing tapered surfaces 524 of arms 520 of the rear cover 104. In this needle shield removal operative orientation, inward deflection of arms 520 of the rear cover 104 is still prevented due to the presence of the safety cap 170 within the automatic injection device 100, thereby preventing rearward axial displacement of the needle guard 110 relative to the housing element 102. It is a particular feature of an embodiment of the present invention, as specifically seen in FIG. 12B, that the safety cap 170 is selectably coupled with the rear cover 104, such that restraining arms 560 of the safety cap 170 are received through openings 530 of the rear cover 104 and protrude forwardly therefrom. The restraining arms 560 of the safety cap 170 are disposed between the shaft 440 of the plunger rod 150 and between arms 520 of the rear cover 104, thereby preventing inward deflection of the arms 520 under axial rearward displacement by the needle guard 110. The leaf spring portions 564 of the restraining arms 560 of the safety cap 170 keep the safety cap 170 coupled with the rear cover 104, since the leaf spring portions 564 are wider than the openings 530 of the rear cover 104.

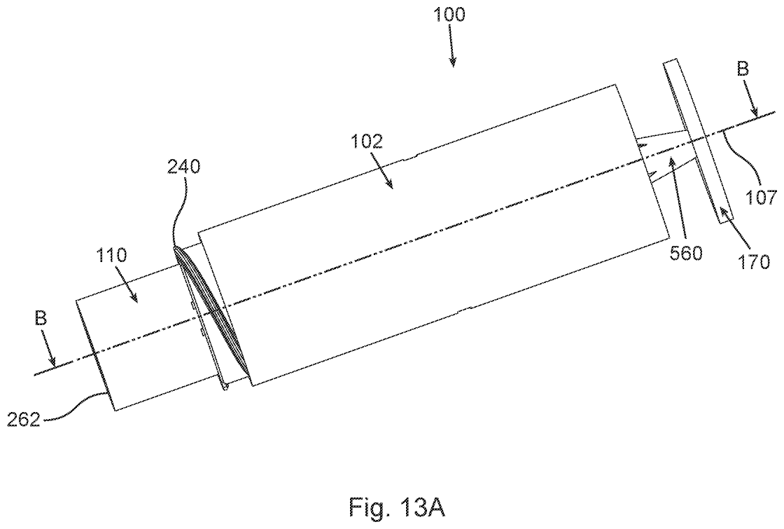

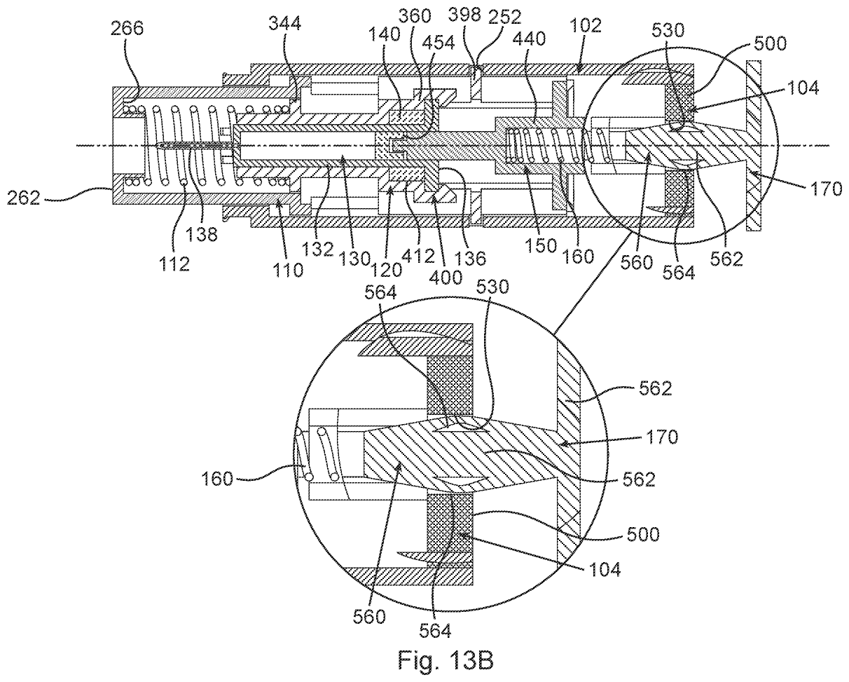

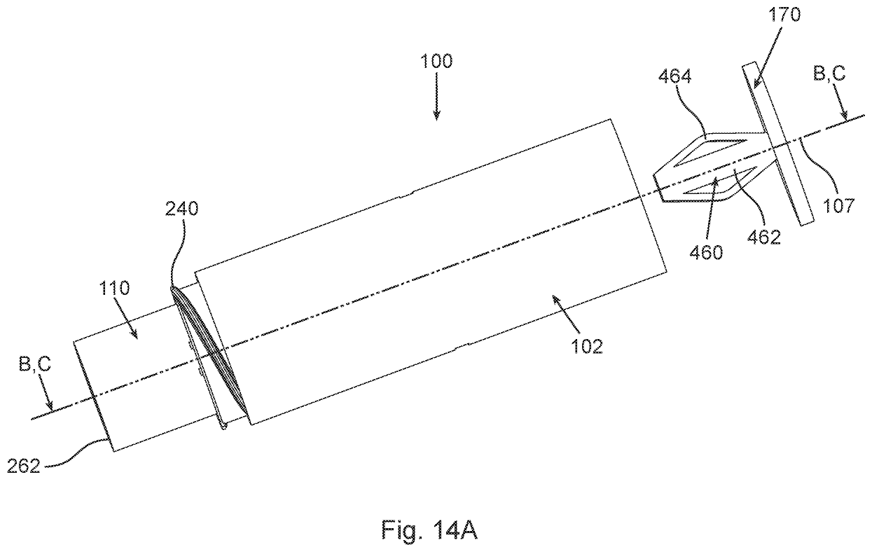

Reference is now made to FIGS. 13A and 13B, which are simplified drawings of the automatic injection device 100 of FIGS. 1A-10D in an intermediate stage of safety cap removal operative orientation, including a simplified perspective view and a sectional view taken along lines B-B in FIG. 13A.

It is appreciated that all spatial relationships between the various components of the automatic injection device 100 remain the same as described hereinabove with respect to the needle shield removal operative orientation illustrated in FIGS. 12A-12C, besides the following spatial relationships:

The user grips the safety cap 170 and axially pulls it rearwardly relative to the housing element 102. When the safety cap 170 is pulled axially rearwardly, the leaf spring portions 564 are tangentially squeezed through openings 530 formed in rearward portion 500 of the rear cover 104 to allow removal of the safety cap 170 from the automatic injection device 100.

FIGS. 13A & 13B illustrate a momentary operative orientation, which occurs during the removal of the safety cap 170 from the automatic injection device 100.

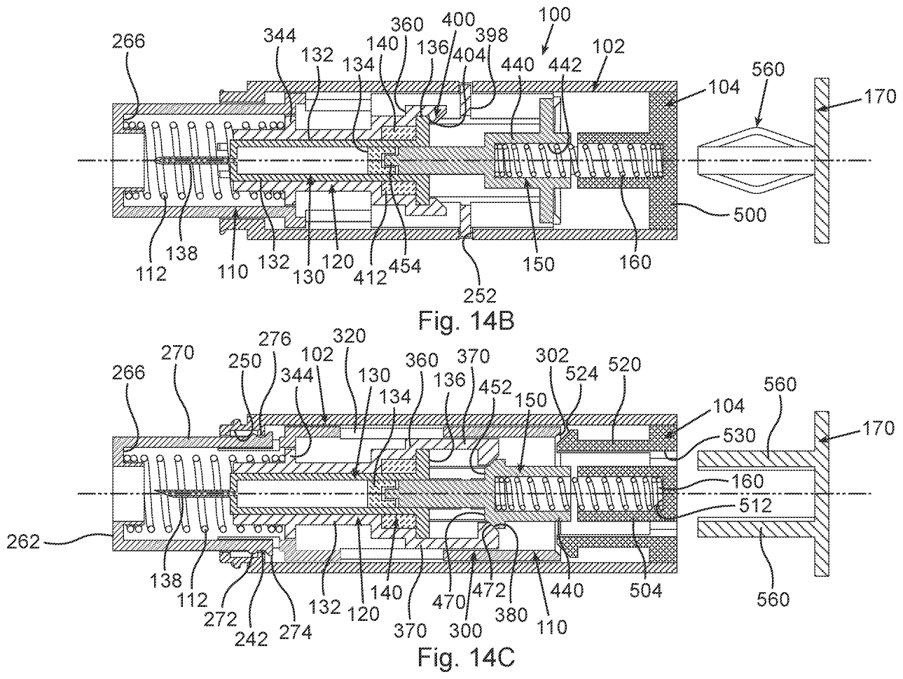

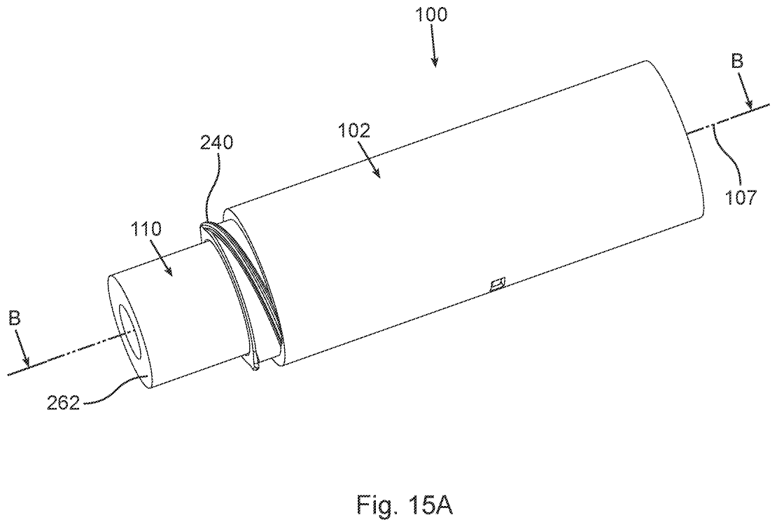

Reference is now made to FIGS. 14A, 14B and 14C, which are simplified drawings of the automatic injection device 100 of FIGS. 1A-10D in an end of safety cap removal operative orientation, including a simplified perspective view and two sectional views taken along orthogonal lines B-B and C-C in FIG. 14A.

It is appreciated that all spatial relationships between the various components of the automatic injection device 100 remain the same as described hereinabove with respect to the intermediate stage of safety cap removal operative orientation illustrated in FIGS. 13A & 13B, besides the following spatial relationships:

The user proceeds pulling the safety cap 170 axially rearwardly relative to the housing element 102. When the safety cap 170 is pulled axially rearwardly, the leaf spring portions 564 are further tangentially squeezed through openings 530 by applying tangential forces on the sides of openings 530 formed in rearward portion 500 of the rear cover 104 to allow full removal of the safety cap 170 from the automatic injection device 100.

It is seen in FIG. 14C that the needle guard 110 remains selectably attached to housing element 102 in this end of safety cap removal operative orientation by means of engagement of the outwardly protruding fingers 272 of snap portions 270 of the needle guard 110 with rearwardly facing shoulder 242 of the housing element 102. Alternatively, the outwardly protruding fingers 272 of the needle guard 110 may be obviated.

It is a particular feature of an embodiment of the present invention that rearward axial displacement of the needle guard 110 relative to the housing element 102 is permitted once the safety cap 107 is fully removed from the rear cover 104. Specifically, when safety cap 170 is removed from the rear cover 104, axial rearward displacement of the needle guard 110 is permitted due to the fact that the arms 520 of the rear cover 104 are now allowed to be inwardly deflected.

It is seen in FIG. 14C that the rearwardmost ends 302 of the mounting arms 300 of needle guard 110 are rearwardly supported against forwardly facing tapered surfaces 524 of arms 520 of the rear cover 104.

It is a particular feature of an embodiment of the present invention that the rearward end of the automatic injection device 100 typically has two lateral openings, namely openings 530 of the rear cover 104, when the safety cap 170 is removed and the forward end of the automatic injection device 100 has one central opening 264 configured for needle penetration therethrough. The different type of openings on both sides of the automatic injection device 100 is beneficial from usability perspective, as user confusion is prevented and there is a clear indication of what side of the automatic injection device 100 should be pressed against the skin for needle penetration therethrough.

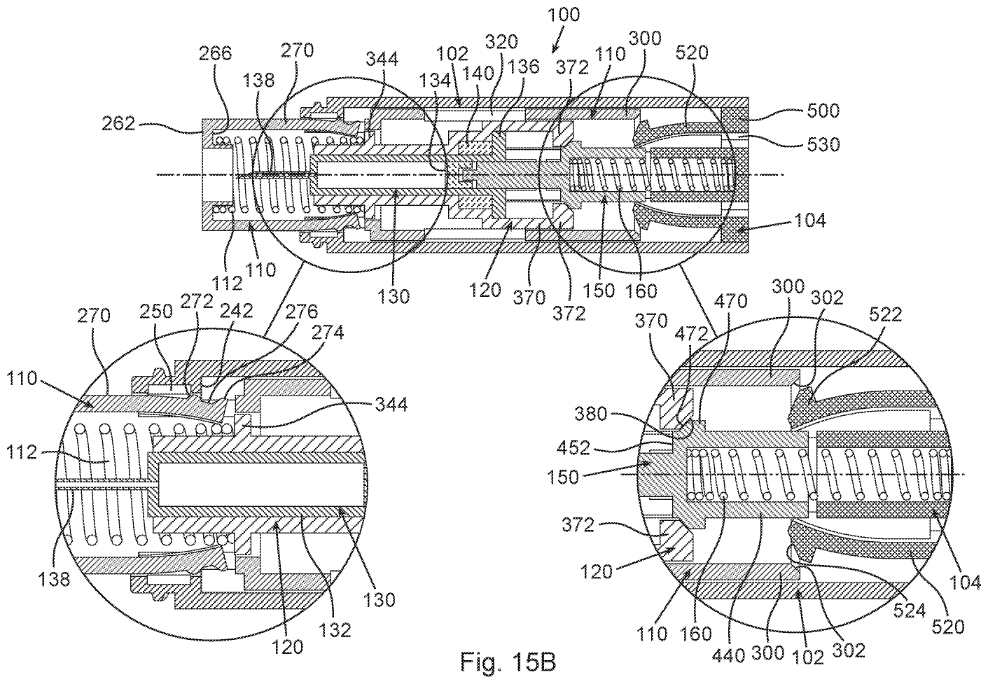

Reference is now made to FIGS. 15A and 15B, which are simplified drawings of the automatic injection device 100 of FIGS. 1A-10D in a needle guard pressing against an injection site operative orientation, including a simplified perspective view and a sectional view taken along lines B-B in FIG. 15A.

It is appreciated that all spatial relationships between the various components of the automatic injection device 100 remain the same as described hereinabove with respect to the end of safety cap removal operative orientation illustrated in FIGS. 14A-14C, besides the following spatial relationships:

The user presses the automatic injection device 100 against an injection site to displace the needle guard 110 axially rearwardly along axis 107 with respect to the remainder of the automatic injection device 100, thus compresses the needle guard spring 112 and thereby initiates manual penetration of the needle 138 into the injection site.

It is noted that the operative orientation illustrated in FIGS. 15A & 15B is a momentary stage, in which the needle guard 110 is shown during the axial rearward displacement thereof, thereby initiating actuation of the automatic injection device 100.

It is seen in FIG. 15B that the needle guard 110 is slightly axially rearwardly displaced relative to the housing element 102, thus the needle guard 110 is no more engaged with the housing element 102.

It is a particular feature of an embodiment of the present invention that the user has to overcome a predetermined trigger force threshold in order to decouple the needle guard 110 from the housing element 102 and thereby displace the needle guard 110 axially rearwardly. The trigger force threshold is preferably within the range of 1 kgf-3.5 kgf. This trigger force is advantageous in that it minimizes the possibility that the user will deter and will not penetrate the needle into the injection site. The trigger force that the user has to apply in order to displace the needle guard 110 rearwardly preferably simultaneously causes an abrupt manual penetration of the needle into the injection site with no opportunity to stop before full needle penetration has occurred.

It is noted that the trigger force is preferably higher than the force of the needle guard spring 112 in combination with the force required for the outwardly protruding finger 272 of the needle guard 110 to jump over the shoulder 242 of the housing element 102, to cause a sufficient penetration depth of the needle 138 in order to inject the medicament within the intra-muscular tissues.

It is noted that in an alternative embodiment of the present invention, the outwardly protruding fingers 272 of the needle guard 110 may be obviated, thus the needle guard 110 is not coupled to the housing element 102 at all. In accordance with this alternative embodiment, the trigger force that the user has to apply in order to displace the needle guard 110 relative to the housing element 102 is created due to friction force between the inner surface of the needle guard 110 and the outer surface of the control unit 120 and additionally due to friction forces created upon engagement of the rearwardmost ends 302 of the mounting arms 300 of the needle guard 110 with tapered surfaces 524 of arms 520 of the rear cover 104. The trigger force threshold is preferably within the range of 1 kgf-3.5 kgf.

It is a particular feature of an embodiment of the present invention that the user preferably applies a substantially constant force up to full penetration of the needle into the desired injection depth. This is achieved due to the friction forces that are present between the needle guard 110 and the control unit 120 along the entire axial rearward displacement of the needle guard 110 relative to the housing element 102. The trigger force that the user has to apply in order to displace the needle guard 110 rearwardly preferably simultaneously causes an abrupt manual penetration of the needle into the injection site with no opportunity to stop before full needle penetration has occurred. The automatic injection device 100 is preferably used for emergency situations, e.g. for injection of epinephrine into the intra-muscular tissue, usually at the outer thigh area of the body, and possibly through layers of closing. The substantially constant force profile of the automatic injection device actuation is beneficial for squeezing the layers of clothing and the soft tissues of the body in order to reach the desired injection depth.

It is particularly seen in FIG. 15B that during decoupling of the needle guard 110 from the housing element 102, the snap portions 270 of the needle guard 110 are inwardly deflected such that the outwardly protruding fingers 272 of the snap portions 270 of the needle guard 110 do not anymore engage the openings 250 within the housing element 102. Outwardly protruding fingers 272 and 274 of the snap portions 270 of the needle guard 110 are now disposed generally rearwardly relative to the rearwardly facing surface 242 of the housing element 102. Alternatively, the outwardly protruding fingers 272 of the needle guard 110 may be obviated.

The needle guard spring 112 is partially compressed in this needle guard pressing against an injection site operative orientation.

It is a particular feature of an embodiment of the present invention that the plunger rod 150 remains prevented from axial forward displacement along longitudinal axis 107 in this needle guard pressing against an injection site operative orientation. The plunger rod 150 is biased axially forwardly under the urge of the injection spring 160, but prevented from such axial forward displacement by engagement with the control unit 120. Specifically, forwardly facing tapered surface 472 of protrusions 470 of the plunger rod 150 are forwardly supported against rearwardly facing tapered surfaces 380 of locking elements 370 of the control unit 120. The locking elements 370 of the control unit 120 are in turn outwardly radially supported against mounting arms 300 of the needle guard 110 and thus are prevented from radial outward deflection and from releasing the plunger rod 150 to be forwardly displaced. The control unit 120 is static relative to the housing element 102, thus the plunger rod 150 is also static relative to the housing element 102 in this needle guard pressing against an injection site operative orientation.

The openings 320 within arms 300 of the needle guard 110 are still forwardly spaced from the radially extending fingers 372 of the locking elements 370 of the control unit 120 in this needle guard pressing against an injection site operative orientation.

It is a particular feature of an embodiment of the present invention that once the user overcomes the trigger force threshold and displaces the needle guard 110 axially rearwardly relative to the housing element 102, axial rearward displacement of the needle guard 110 translated into inward radial deflection of arms 520 of the rear cover 104, due to engagement of the rearwardmost ends 302 of the mounting arms 300 of needle guard 110 with forwardly facing tapered surfaces 524 of arms 520 of the rear cover 104. It is specifically seen in FIG. 15B that arms 520 of the rear cover during this needle guard pressing against an injection site operative orientation are radially inwardly deflected towards shaft 440 of the plunger rod 150.

It is particularly seen that in this illustrative momentary operative orientation of needle guard pressing against an injection site the needle 138 is still protected within the needle guard 110.

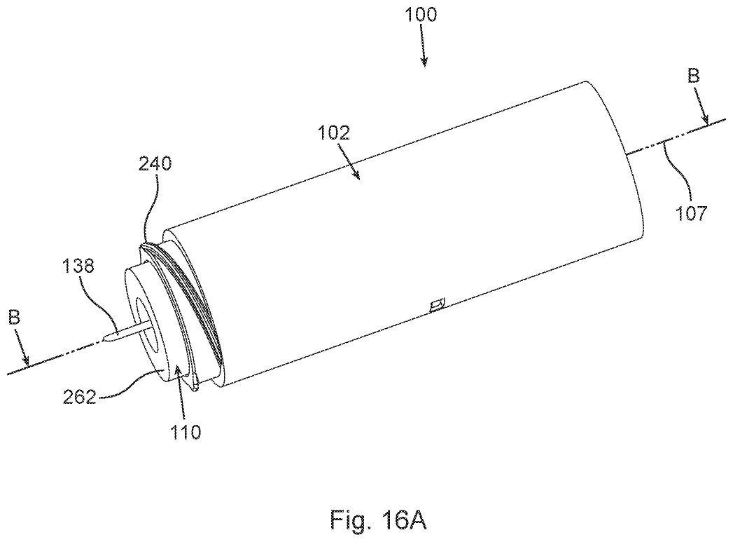

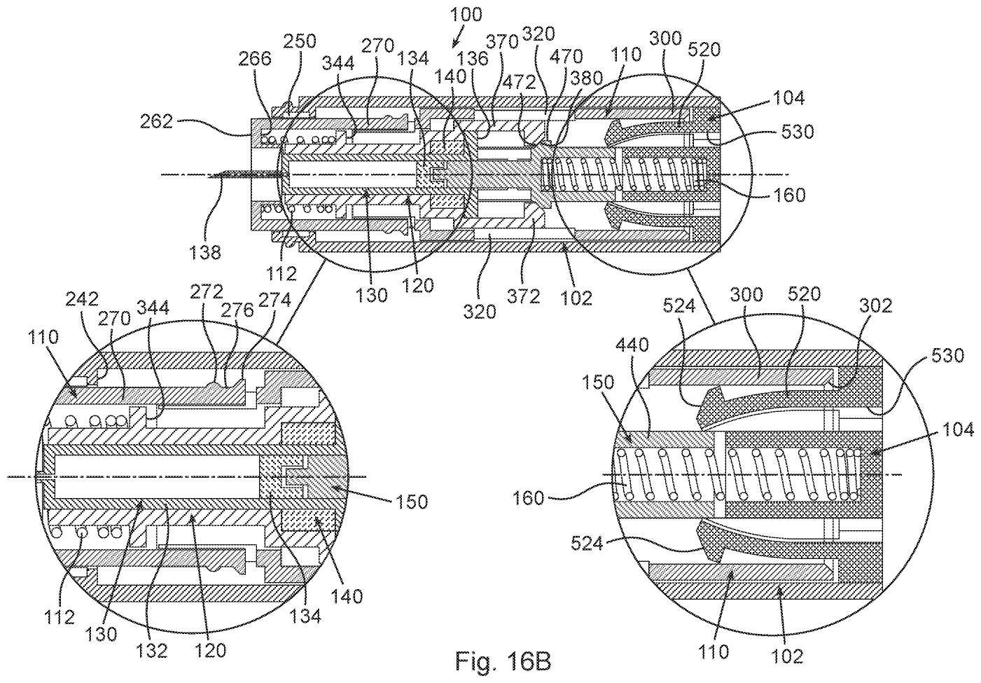

Reference is now made to FIGS. 16A and 16B, which are simplified drawings of the automatic injection device 100 of FIGS. 1A-10D in a needle insertion operative orientation, including a simplified perspective view and a sectional view taken along lines B-B in FIG. 16A.

It is appreciated that all spatial relationships between the various components of the automatic injection device 100 remain the same as described hereinabove with respect to the needle guard pressing against an injection site operative orientation illustrated in FIGS. 15A & 15B, besides the following spatial relationships:

The user continues pressing the automatic injection device 100 against the injection site to fully displace the needle guard 110 axially rearwardly along axis 107 with respect to the remainder of the automatic injection device 100, thus fully compresses the needle guard spring 112 and thereby manually penetrating the needle 138 into the injection site. It is noted that the automatic injection device 100 is still in the process of actuation, as illustrated in FIGS. 16A and 16B.

It is seen in FIG. 16B that the needle guard 110 is axially rearwardly displaced relative to the housing element 102, thus the needle guard 110 is no more engaged with the housing element 102.

It is particularly seen in FIG. 16B that the snap portions 270 of the needle guard 110 are no more inwardly deflected, rather they return to their at rest position. Outwardly protruding fingers 272 and 274 of the snap portions 270 of the needle guard 110 are now disposed generally more rearwardly relative to the rearwardly facing surface 242 of the housing, in comparison to their position as illustrated in FIGS. 15A & 15B.

It is a particular feature of an embodiment of the present invention that the radially extending fingers 372 of the locking elements 370 of the control unit 120 in this needle insertion operative orientation are positioned in front of the openings 320 within arms 300 of the needle guard 110 and therefore are permitted to deflect radially outwardly under the urge of the injection spring 160, which biases the plunger rod axially forwardly. It is particularly seen that in this illustrative momentary operative orientation of needle insertion the locking elements 370 of the control unit 120 are permitted to deflect radially outwardly, but in the illustrations shown in FIGS. 16A & 16B, the locking elements 370 are not yet radially outwardly deflected.

It is seen in FIG. 16B that the needle guard 110 is now fully retracted axially rearwardly, thereby causing radial inward deflection of the arms 520 of the rear cover 104.

In FIGS. 16A & 16B a momentary operative orientation is shown in which the plunger rod 150 still engages the control unit 120 and is not yet axially forwardly displaced. Specifically, forwardly facing tapered surface 472 of protrusions 470 of the plunger rod 150 are forwardly supported against rearwardly facing tapered surfaces 380 of locking elements 370 of the control unit 120. The locking elements 370 of the control unit 120 are not prevented anymore from radial outward deflection into openings 320 of mounting arms 300 of the needle guard 110.

It is seen in FIG. 16B that the needle guard 110 protrudes forwardly from the housing element 102 to a second longitudinal extent, which is lesser than the first longitudinal extent, as shown in the storage operative orientation and illustrated in FIGS. 11A-11C.

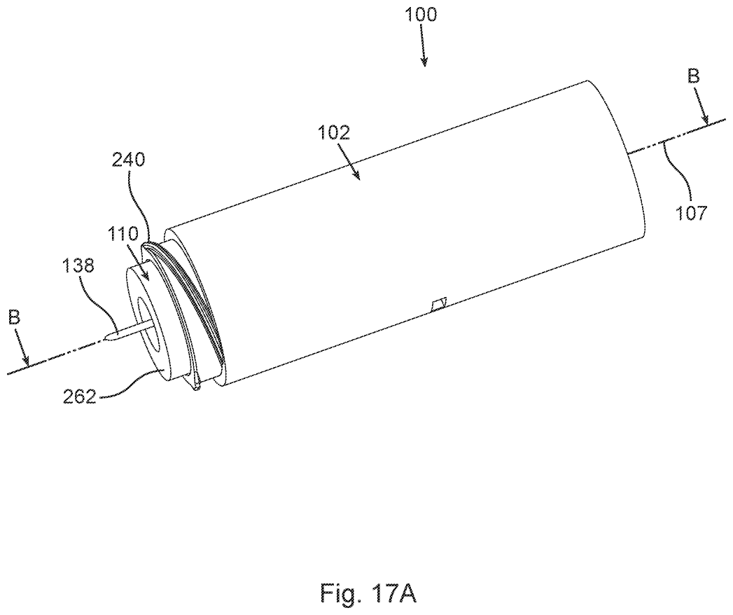

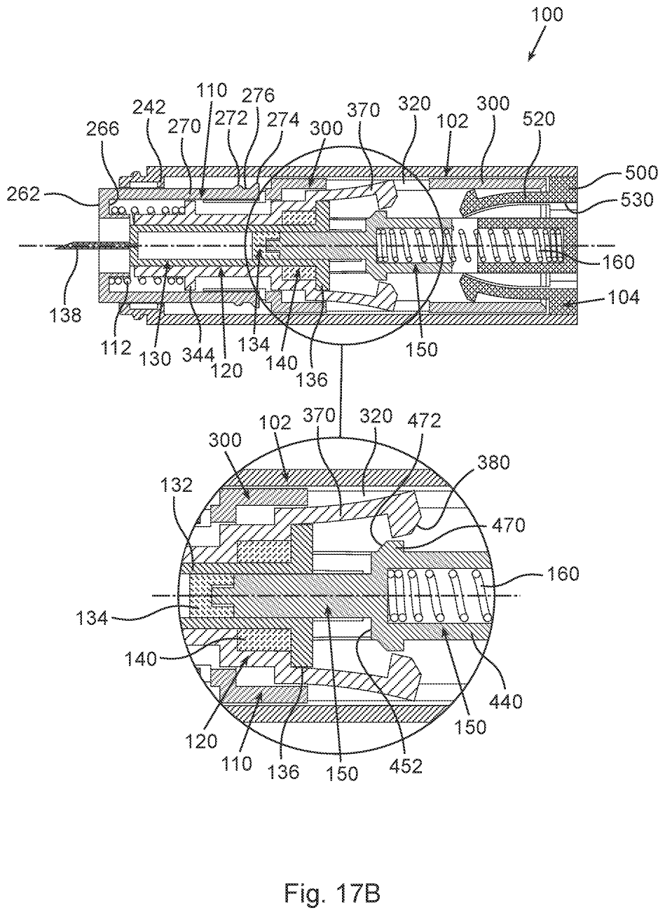

Reference is now made to FIGS. 17A and 17B, which are simplified drawings of the automatic injection device of FIGS. 1A-10D in an injection initiation operative orientation, including a simplified perspective view and a sectional view taken along lines B-B in FIG. 17A.

It is appreciated that all spatial relationships between the various components of the automatic injection device 100 remain the same as described hereinabove with respect to the needle insertion operative orientation illustrated in FIGS. 16A & 16B, besides the following spatial relationships:

The user continues pressing the automatic injection device 100 against the injection site to effect initiation of ejection of medicament from the syringe barrel 132 via needle 138 into the injection site. The needle guard 110 remains fully rearwardly retracted and the needle 138 remains within the injection site. It is noted that the automatic injection device 100 is illustrated in an actuated operative orientation, as illustrated in FIGS. 17A and 17B.

It is a particular feature of an embodiment of the present invention that the locking elements 370 of the control unit 120 are now radially outwardly deflected into openings 320 of mounting arms 300 of the needle guard 110 to allow the plunger rod 150 to slide over the locking elements 370 of the control unit 120 under the bias of the injection spring 160 and be axially forwardly displaced relative to the syringe barrel 132 to effect ejection of medicament out of the syringe barrel 132.

It is noted that the axial position of the openings 320 of mounting arms 300 of the needle guard 110 along the longitudinal extent of the needle guard 100 defines when the automatic injection device 100 is actuated and thereby defines when the injection is initiated. Specifically, the injection is initiated only when the locking elements 370 of the control unit 120 are deflected outwardly and engage the openings 320 of the needle guard 110. The extent of axial rearward displacement of the needle guard 110 relative to the housing element 102 that is required in order to allow radial deflection of the locking elements 370 can be utilized to adjust the needle penetration depth at time of injection initiation. For example, when the extent of the axial rearward displacement of the needle guard 110 relative to the housing element 102 is smaller, the needle penetration depth is smaller, thus such a configuration may be more suitable for kids. Alternatively, when the extent of the axial rearward displacement of the needle guard 110 relative to the housing element 102 is larger, the needle penetration depth is larger, thus such configuration may be more suitable for adults. The extent of the axial rearward displacement of the needle guard 110 relative to the housing element 102, which provides initiation of injection, is defined by the longitudinal extent of the openings 320 of the needle guard 110.

It is a particular feature of an embodiment of the present invention that the plunger rod 150 is now slightly displaced axially forwardly to engage piston 134. The plunger rod 150 is now axially forwardly displaced along longitudinal axis 107 under the bias of the injection spring 160. It is specifically seen in FIG. 17B that the plunger 150 does not engage the control unit 120 anymore in this injection initiation operative orientation. Specifically, forwardly facing tapered surface 472 of protrusions 470 of the plunger rod 150 are not supported against rearwardly facing tapered surfaces 380 of locking elements 370 of the control unit 120 anymore.

It is seen in FIG. 17B that the injection spring 160 is now disposed in a partially released operative orientation.

It is noted that in this injection initiation operative orientation the forward end 452 of shaft 440 of the plunger rod 150 is slightly less rearwardly spaced from the flange 136 of the syringe barrel 132 in comparison with its position in storage operative orientation, as illustrated in FIGS. 11A-11C, and the distance between the forward end 452 and the flange 136 defines the amount of medicament to be ejected from the syringe barrel 132.

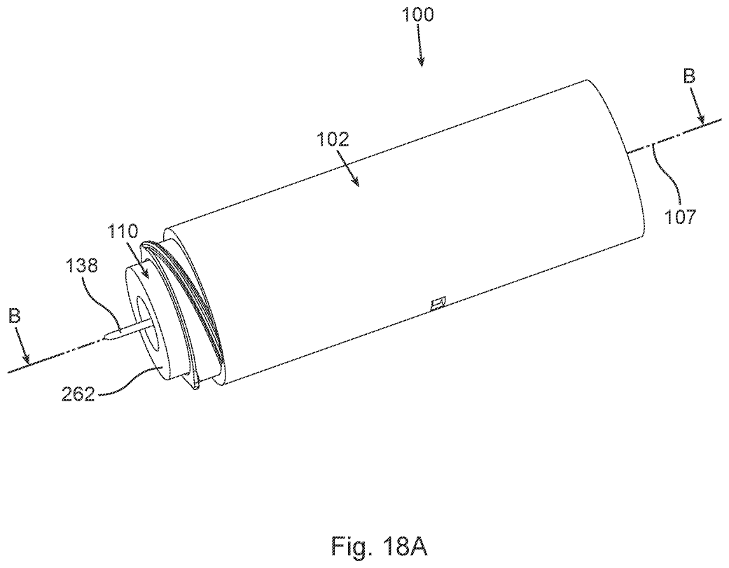

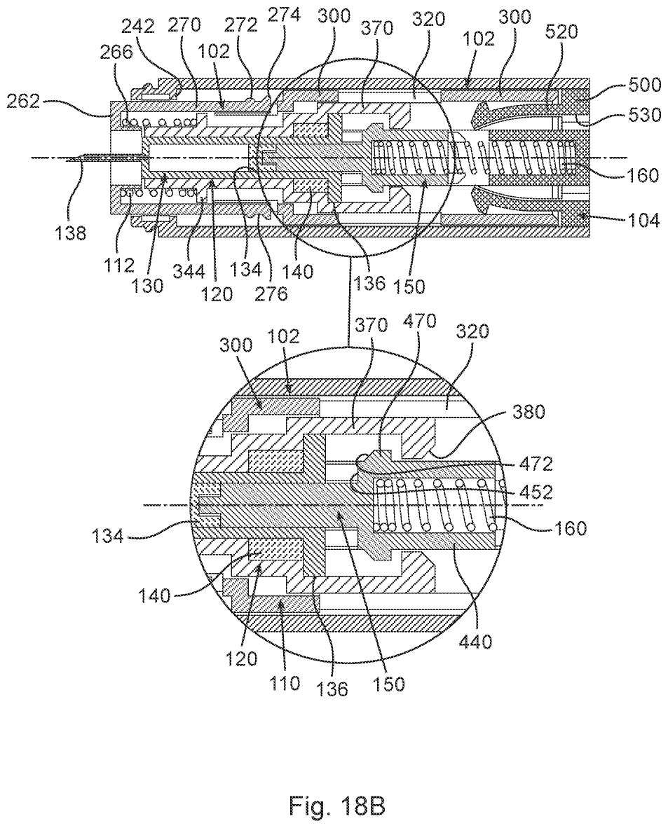

Reference is now made to FIGS. 18A and 18B, which are simplified drawings of the automatic injection device 100 of FIGS. 1A-10D in an injection operative orientation, including a simplified perspective view and a sectional view taken along lines B-B in FIG. 18A.

It is appreciated that all spatial relationships between the various components of the automatic injection device 100 remain the same as described hereinabove with respect to the injection initiation operative orientation illustrated in FIGS. 17A & 17B, besides the following spatial relationships:

The user continues pressing the automatic injection device 100 against the injection site to effect initiation of ejection of medicament from the syringe barrel 132 via needle 138 into the injection site. The needle guard 110 remains fully rearwardly retracted and the needle 138 remains within the injection site. The plunger rod 150 continues its forward axial displacement relative to the housing element 102 and relative to the syringe assembly 130 under the urge of the injection spring 160.

The locking elements 370 of the control unit 120 now return to their at rest operative orientation in front of openings 320 of mounting arms 300 of the needle guard 110.

It is a particular feature of an embodiment of the present invention that the plunger rod 150 is now displaced more axially forwardly along longitudinal axis 107, in comparison to the injection initiation operative orientation, as illustrated in FIGS. 17A & 17B. It is specifically seen in FIGS. 18A & 18B that the plunger 150 does not engage the control unit 120 anymore in this injection operative orientation. Specifically, forwardly facing tapered surface 472 of protrusions 470 of the plunger rod 150 are now forwardly spaced relative to the rearwardly facing tapered surfaces 380 of locking elements 370 of the control unit 120.

It is noted that in this injection operative orientation the forward end 452 of shaft 440 of the plunger rod 150 is less rearwardly spaced from the flange 136 of the syringe barrel 132 in comparison with its position in injection initiation operative orientation, as illustrated in FIGS. 17A & 17B.

It is seen in FIG. 18B that the injection spring 160 is now disposed in a more released operative orientation in comparison to its orientation in injection initiation, as illustrated in FIGS. 17A & 17B.

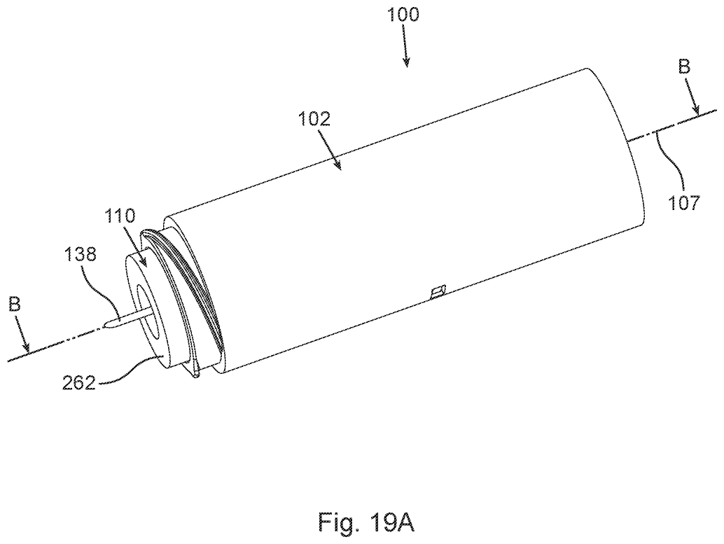

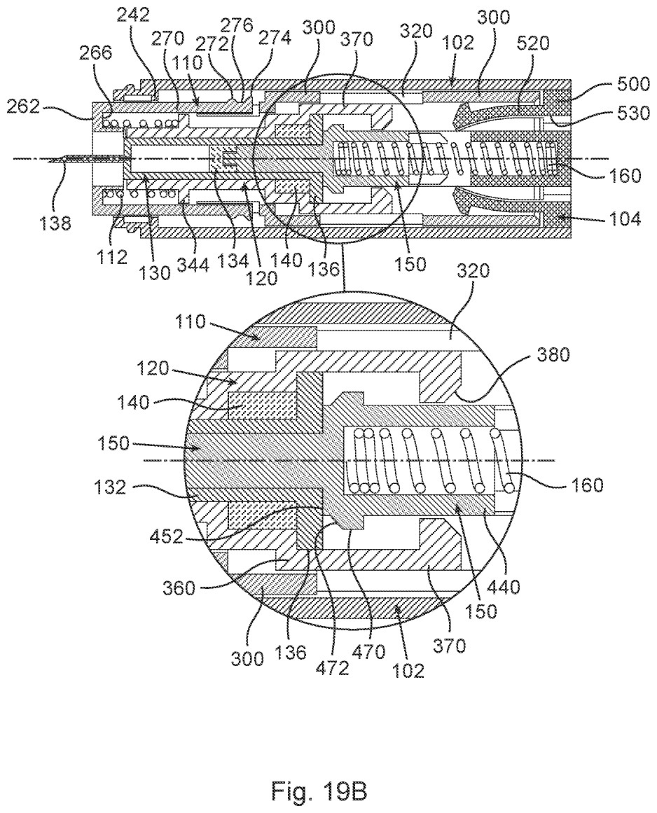

Reference is now made to FIGS. 19A and 19B, which are simplified drawings of the automatic injection device 100 of FIGS. 1A-10D in an end of injection operative orientation, including a simplified perspective view and a sectional view taken along lines B-B in FIG. 19A.

It is appreciated that all spatial relationships between the various components of the automatic injection device 100 remain the same as described hereinabove with respect to the injection operative orientation illustrated in FIGS. 18A & 18B, besides the following spatial relationships:

The user continues pressing the automatic injection device 100 against the injection site to effect initiation of ejection of medicament from the syringe barrel 132 via needle 138 into the injection site. The needle guard 110 remains fully rearwardly retracted and the needle 138 remains within the injection site. The plunger rod 150 continues its forward axial displacement relative to the housing element 102 and relative to the syringe assembly 130 under the urge of the injection spring 160 up to ejection of a predetermined amount of medicament from the syringe barrel 132.

It is a particular feature of an embodiment of the present invention that in this end of injection operative orientation the plunger rod 150 is displaced more axially forwardly along longitudinal axis 107 up to its forwardmost position, specifically up to engagement of a portion of the plunger rod 150 with the flange 136 of the syringe barrel 132. Specifically, as seen in FIG. 19B, the forward end 452 of shaft 440 of the plunger rod 150 now abuts the flange 136 of the syringe barrel 132.

It is a particular feature of an embodiment of the present invention that the dampening element 140 slightly protrudes rearwardly from rearward annular wall 360 of the control unit 120, thereby partially absorbing the forces created during relative displacement of the plunger rod 150 and the syringe barrel 132. Specifically, upon full forward axial displacement of the plunger rod 150 relative to the housing element 102, the forward end 452 of the shaft 440 of the plunger rod 150 engages the flange 136 of the syringe barrel 132. Since the dampening element 140 slightly protrudes rearwardly from the annular wall 360 of the control unit 120, it is configured to partially absorb the forces exerted on the flange 136 of the syringe barrel 132 during its engagement with the forward end 452 of the plunger rod 150 and thereby prevent or minimize the risk of breakage of the flange 136.

It is seen in FIG. 19B that the injection spring 160 is now disposed in a released operative orientation.

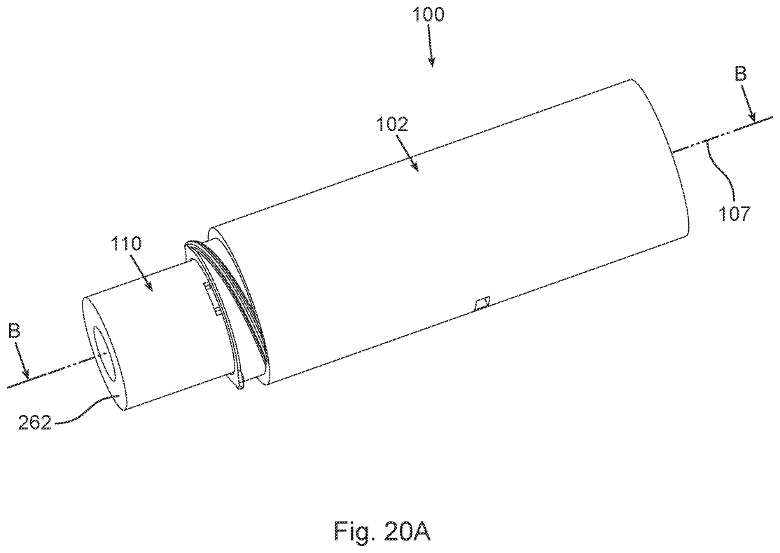

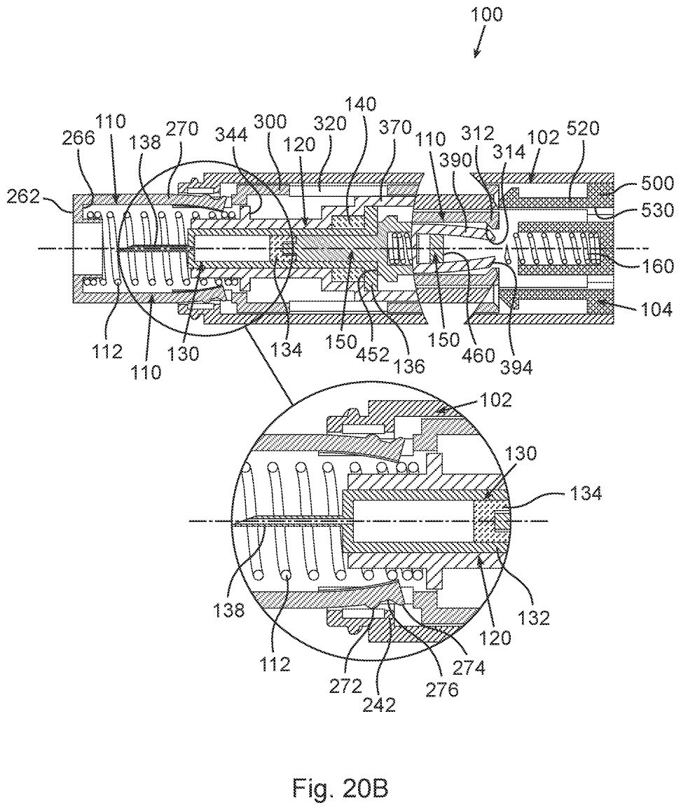

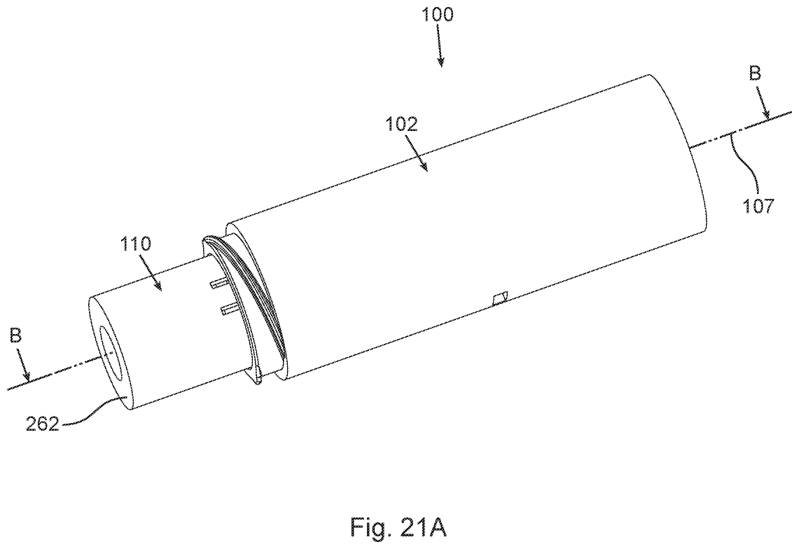

Reference is now made to FIGS. 20A and 20B, which are simplified drawings of the automatic injection device 100 of FIGS. 1A-10D in a removal from injection site operative orientation, including a simplified perspective view and a sectional view taken along lines B-B in FIG. 20A, FIG. 20B is partially cut-out to clearly show the internal components of the automatic injection device 100 of FIGS. 1A-10D.

It is appreciated that all spatial relationships between the various components of the automatic injection device 100 remain the same as described hereinabove with respect to the end of injection operative orientation illustrated in FIGS. 19A & 19B, besides the following spatial relationships:

The user removes the automatic injection device 100 from the injection site, thereby urging axial forward displacement of the needle guard 110 relative to the housing element 102 in order to cover and protect the needle 138. In this illustrative operative orientation, the needle guard 110 is shown in a momentary position during the axial forward displacement thereof, but when the needle guard 110 is not yet fully deployed over the needle 138.

The plunger rod 150 remains in its forwardmost position when the forward end 452 of the shaft 440 of the plunger rod 150 abuts the flange 136 of the syringe barrel 132.

It is a particular feature of an embodiment of the present invention that when the plunger rod 150 is disposed at its forward position, it does not support the two pairs of ribs 390 of the control unit 120 anymore, thus the ribs 390 are allowed to be inwardly deflected towards each other and thereby allow axial forward displacement of the needle guard 110 relative to housing element 102 under the urge of the needle guard spring 112 once the user releases the automatic injection device 100 from the injection site.

It is specifically seen in FIG. 20B the forwardly facing tapered edges 314 of inwardly facing protrusions 312 of the needle guard 110 abut the rearward tapered edges 394 of two support ribs 390 of the control unit 120 and since the plunger rod 150 along with the protrusions 460 is now displaced axially forwardly, protrusions 460 do not prevent the support ribs 390 of the control unit 120 from inward deflection under the axial forward bias of the needle guard spring 112, thereby permitting forward axial displacement of the needle guard 110 relative to the housing element 102.

It is seen that in this momentary illustrative operative orientation the needle guard 110 is not fully displaced axially forwardly, but that the snap portions 270 of the needle guard 110 started to deflect radially inwardly towards each other due to engagement thereof with the rearwardly facing shoulder 242 of the housing element 102.

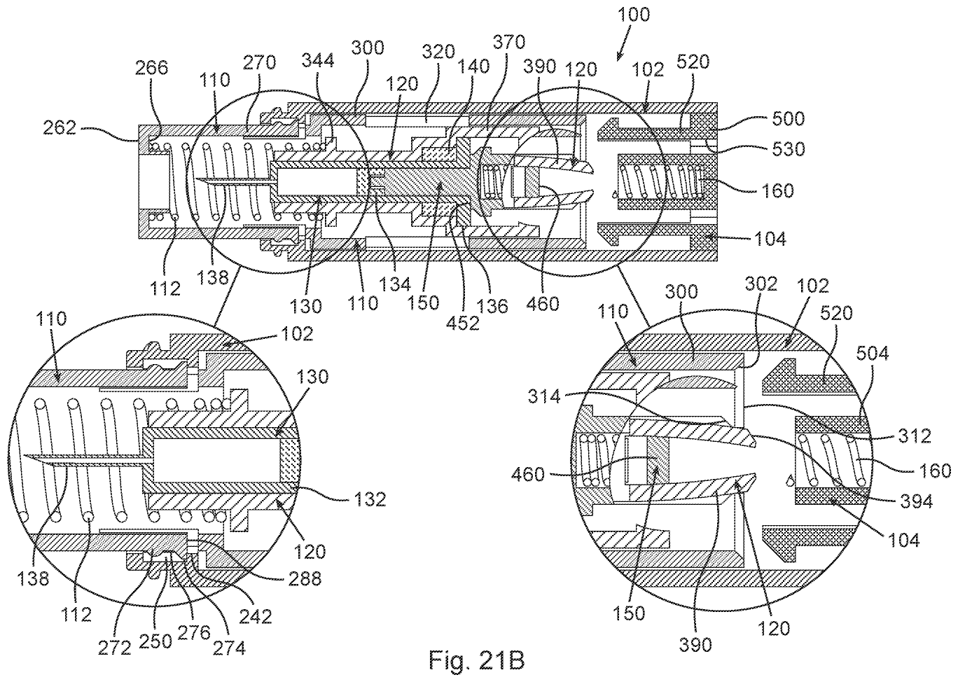

Reference is now made to FIGS. 21A and 21B, which are simplified drawings of the automatic injection device 100 of FIGS. 1A-10D in a needle protection operative orientation, including a simplified perspective view and a sectional view taken along lines B-B in FIG. 21A, FIG. 21B is partially cut-out to clearly show the internal components of the automatic injection device 100 of FIGS. 1A-10D.

It is appreciated that all spatial relationships between the various components of the automatic injection device 100 remain the same as described hereinabove with respect to the removal from injection site operative orientation illustrated in FIGS. 20A & 20B, besides the following spatial relationships:

The user fully removes the automatic injection device 100 from the injection site, thereby urging axial forward displacement of the needle guard 110 relative to the housing element 102 in order to cover and protect the needle 138. In this operative orientation, the needle guard 110 is shown in a fully deployed orientation at its forwardmost position relative to the housing element 102, where the needle guard 110 is locked relative to housing element 102 and the needle 138 is protected.

It is noted that the needle guard 110 now protrudes forwardly from the housing element 102 to a second longitudinal extent, which is greater than the first longitudinal mentioned with reference to FIGS. 11A-11C illustrating the automatic injection device 100 is the storage operative orientation.

It is a particular feature of an embodiment of the present invention that when the plunger rod 150 is disposed at its forward position, it does not support the two pairs of ribs 390 of the control unit 120 anymore, thus the ribs 390 are allowed to be inwardly deflected towards each other and thereby allow further axial forward displacement of the needle guard 110 relative to housing element 102 under the urge of the needle guard spring 112 once the user releases the automatic injection device 100 from the injection site.

It is specifically seen in FIG. 21B that the forwardly facing tapered edges 314 of inwardly facing protrusions 312 of the needle guard 110 are now forwardly spaced relative to the rearward tapered edges 394 of two support ribs 390 of the control unit 120 and the support ribs 390 of the control unit 120 are inwardly deflected under the axial forward bias of the needle guard 110, thereby permitting further forward axial displacement of the needle guard 110 relative to the housing element 102.

It is a particular feature of an embodiment of the present invention that in this needle protection operative orientation the needle guard 110 is fully displaced axially forwardly and is locked relative to the housing element 102. It is specifically seen in FIG. 21B that the snap portions 270 of the needle guard 110 slide over the rearwardly facing shoulder 242 of the housing element 102, such that both outwardly protruding fingers 272 and 274 of the snap portions 270 of the needle guard 110 are now disposed within openings 250 of the housing element 102 and the rearwardly facing shoulder 288 of the outwardly protruding finger 274 is now rearwardly supported by the rearwardly facing shoulder 242 of the housing element 102, such that rearward axial displacement of the needle guard 110 relative to the housing element 102 and exposure of needle 138 is prevented in this needle protection orientation.

It is noted that both the needle guard spring 112 and the injection spring 160 are disposed in their released state in this needle protection operative orientation.

This invention generally relates to an automatic injection device for parenteral administration of substances (e.g., a medication) to a living organism (human or animal). The administration may be delivered into the subcutaneous tissue or intramuscularly.

It is appreciated that in accordance with an embodiment of the present invention the medicament is enclosed in a pre-filled syringe, but it can alternatively be used with other drug enclosures such as vials or ampoules, where a vial adaptor or an ampoule adaptor is used to reconstitute, mix, or pump the drug into the syringe prior to injection. The pre-filled syringe can be either a conventional one chambered pre-filled syringe with a ready-to-inject liquid form drug, or it can be a multiple-chambered pre-filled syringe.

It will be appreciated by persons skilled in the art that the present invention is not limited by what has been particularly shown and described hereinabove. Rather the scope of the present invention includes both combinations and sub-combinations of various features described hereinabove as well as variations and modifications thereof which are not in the prior art.

1.-154. (canceled)

155. An automatic injection device for use with a syringe including at least one syringe piston and a needle coupled to a forward end thereof, comprising:
a housing element arranged along a longitudinal axis and having a forward end and a rearward end;
at least one resilient element arranged to be located within said housing element;
a needle guard slidably positionable with respect to said housing element;
a control unit configured to fixedly retain said syringe therewithin and configured to be fixedly coupled with said housing element;
a plunger rod biased axially forwardly under an urge of said at least one resilient element and configured to engage the control unit in a storage operative orientation, in which said plunger rod is prevented from forward axial displacement relative to said control unit under said urge of said at least one resilient element; and wherein upon axial rearward displacement of said needle guard with respect to said housing element, said plunger rod is disengaged from said control unit and is permitted to be axially forwardly displaced relative to said control unit under said urge of said at least one resilient element,
and wherein said control unit has at least one locking portion and wherein said plunger rod is configured to engage the at least one locking portion in said storage operative orientation and upon said axial rearward displacement of said needle guard with respect to said housing element, said plunger rod is disengaged from said at least one locking portion.

156. The automatic injection device of claim 155, also comprising a rear cover, which is fixedly attached to said rearward end of said housing element and wherein a portion thereof is configured to be radially deflected upon said axial rearward displacement of said needle guard.

157. The automatic injection device of claim 156, and wherein said axial rearward displacement of said needle guard is prevented by engagement thereof with said rear cover.

158. The automatic injection device of claim 155, and wherein said at least one locking portion is radially deflectable.

159. The automatic injection device of claim 155, and wherein said plunger rod has at least one protrusion, which is forwardly supported against said at least one locking portion of said control unit in said storage operative orientation, and wherein said at least one locking portion is outwardly radially supported against a portion of said needle guard and thus prevented from radial outward deflection and from releasing the plunger rod to be forwardly displaced in said storage operative orientation.

160. The automatic injection device of claim 155, and wherein displacement of said needle guard relative to said housing element requires overcoming a predetermined trigger force threshold.

161. The automatic injection device of claim 155, and also comprising a needle shield remover, configured to be releasably coupled to said housing element; and wherein said needle guard engages a portion of the needle shield remover in a storage operative orientation, thereby preventing said needle guard from being axially forwardly displaced relative to said housing element.

162. An automatic injection device for use with a syringe including at least one syringe piston and a needle coupled to a forward end thereof, comprising:
a housing element arranged along a longitudinal axis and having a forward end and a rearward end;
at least one resilient element arranged to be located within said housing element;
a needle guard slidably positionable with respect to said housing element, said needle guard has at least one opening;
a control unit configured to be fixedly coupled with said housing element, said control unit has at least one locking portion;
a plunger rod biased axially forwardly under an urge of said at least one resilient element and configured to axially forwardly displace said piston relative to the syringe upon actuation of said automatic injection device; and wherein said locking portion is radially supported by said needle guard in a storage operative orientation and upon axial rearward displacement of said needle guard with respect to said housing element, said at least one locking portion is inserted into said at least one opening of said needle guard, thereby actuating the automatic injection device and allowing axial forward displacement of said plunger rod relative to said syringe.

163. The automatic injection device of claim 162, and wherein said plunger rod is prevented from forward axial displacement relative to said control unit under said urge of said at least one resilient element in said storage operative orientation.

164. The automatic injection device of claim 162, and wherein upon axial rearward displacement of said needle guard with respect to said housing element, said plunger rod is disengaged from said control unit and is permitted to be axially forwardly displaced relative to said control unit under said urge of said at least one resilient element.

165. The automatic injection device of claim 162, and wherein displacement of said needle guard relative to said housing element requires overcoming a predetermined trigger force threshold.

166. The automatic injection device of claim 162, also comprising a rear cover, which is fixedly attached to said rearward end of said housing element and wherein a portion thereof is configured to be radially deflected upon said axial rearward displacement of said needle guard.

167. The automatic injection device of claim 162, and wherein said plunger rod has at least one protrusion, which is forwardly supported against said at least one locking portion of said control unit in said storage operative orientation.

168. An automatic injection device for use with a syringe including at least one syringe piston and a needle coupled to a forward end thereof, comprising:
a housing element arranged along a longitudinal axis and having a forward end and a rearward end;
at least one resilient element arranged to be located within said housing element;
a needle guard slidably positionable with respect to said housing element;
a control unit configured to be fixedly coupled with said housing element, said control unit has at least one locking portion;
a plunger rod biased axially forwardly under an urge of said at least one resilient element and configured to axially forwardly displace said piston relative to the syringe upon actuation of said automatic injection device; said plunger rod has at least one lock engaging portion and wherein said at least one lock engaging portion is forwardly supported by said at least one locking portion in a storage operative orientation and upon axial rearward displacement of said needle guard with respect to said housing element, said at least one locking portion disengages from said at least one lock engaging portion, thereby actuating the automatic injection device and allowing axial forward displacement of said plunger rod relative to said syringe.

169. The automatic injection device of claim 168, also comprising a rear cover, which is fixedly attached to said rearward end of said housing element and wherein a portion thereof is configured to be radially deflected upon said axial rearward displacement of said needle guard.

170. The automatic injection device of claim 169, and wherein said at least one locking portion is radially deflectable.

171. The automatic injection device of claim 169, also comprising a dampening element between said control unit and said syringe, which is configured to partially absorb the forces created during relative displacement of the plunger rod and the syringe.

172. The automatic injection device of claim 169, and wherein said at least one locking portion is outwardly radially supported against a portion of said needle guard and thus prevented from radial outward deflection and from releasing the plunger rod to be forwardly displaced in said storage operative orientation.

173. The automatic injection device of claim 169, and wherein displacement of said needle guard relative to said housing element requires overcoming a predetermined trigger force threshold.

174. The automatic injection device of claim 169, and also comprising a needle shield remover, configured to be releasably coupled to said housing element; and wherein said needle guard engages a portion of the needle shield remover in a storage operative orientation, thereby preventing said needle guard from being axially forwardly displaced relative to said housing element.
